SEQUENCE LISTING (33 sequences)

SEQ ID NO: 1 - DNA, 36 bp, Artificial Sequence
  Description of Artificial Sequence Synthetic oligonucleotide
caggcagaggcttataaaaatcctcgccaacagctt

SEQ ID NO: 2 - DNA, 26 bp, Artificial Sequence
  Description of Artificial Sequence Synthetic oligonucleotide
ggtggcagcagccaactcagcttcct

SEQ ID NO: 3 - DNA, 24 bp, Artificial Sequence
  Description of Artificial Sequence Synthetic oligonucleotide
gattctcatgataagctacgccga

SEQ ID NO: 4 - DNA, 5837 bp, Thermococcus litoralis
gaattcgcgataaaatctattttcttcctccatttttcaatttcaaaaacgtaagcatgagccaaacctctcgccctttctctgtccttcccgctaaccctcttgaaaactctctccaaagcattttttgatgaaagctcacgctcctctatgagggtcagtatatctgcaatgagttcgtgaagggttattctgtagaacaactccatgattttcgatttggatgggggtttaaaaatttggcggaacttttatttaatttgaactccagtttatatctggtggtatttatgatactggacactgattacataacaaaagatggcaagcctataatccgaatttttaagaaagagaacggggagtttaaaatagaacttgaccctcattttcagccctatatatatgctcttctcaaagatgactccgctattgaggagataaaggcaataaagggcgagagacatggaaaaactgtgagagtgctcgatgcagtgaaagtcaggaaaaaatttttgggaagggaagttgaagtctggaagctcattttcgagcatccccaagacgttccagctatgcggggcaaaataagggaacatccagctgtggttgacatttacgaatatgacataccctttgccaagcgttatctcatagacaagggcttgattcccatggagggagacgaggagcttaagctccttgcctttgatattgaaacgttttatcatgagggagatgaatttggaaagggcgagataataatgattagttatgccgatgaagaagaggccagagtaatcacatggaaaaatatcgatttgccgtatgtcgatgttgtgtccaatgaaagagaaatgataaagcgttttgttcaagttgttaaagaaaaagaccccgatgtgataataacttacaatggggacaattttgatttgccgtatctcataaaacgggcagaaaagctgggagttcggcttgtcttaggaagggacaaagaacatcccgaacccaagattcagaggatgggtgatagttttgctgtggaaatcaagggtagaatccactttgatcttttcccagttgtgcgaaggacgataaacctcccaacgtatacgcttgaggcagtttatgaagcagttttaggaaaaaccaaaagcaaattaggagcagaggaaattgccgctatatgggaaacagaagaaagcatgaaaaaactagcccagtactcaatggaagatgctagggcaacgtatgagctcgggaaggaattcttccccatggaagctgagctggcaaagctgataggtcaaagtgtatgggacgtctcgagatcaagcaccggcaacctcgtggagtggtatcttttaagggtggcatacgcgaggaatgaacttgcaccgaacaaacctgatgaggaagagtataaacggcgcttaagaacaacttacctgggaggatatgtaaaagagccagaaaaaggtttgtgggaaaatatcatttatttggatttccgcagtctgtacccttcaataatagttactcacaacgtatccccagatacccttgaaaaagagggctgtaagaattacgatgttgctccgatagtaggatataggttctgcaaggactttccgggctttattccctccatactcggggacttaattgcaatgaggcaagatataaagaagaaaatgaaatccacaattgacccgatcgaaaagaaaatgctcgattataggcaaagggctattaaattgcttgcaaacagcatcttacccaacgagtggttaccaataattgaaaatggagaaataaaattcgtgaaaattggcgagtttataaactcttacatggaaaaacagaaggaaaacgttaaaacagtagagaatactgaagttctcgaagtaaacaacctttttgcattctcattcaacaaaaaaatcaaagaaagtgaagtcaaaaaagtcaaagccctcataagacataagtataaagggaaagcttatgagattcagcttagctctggtagaaaaattaacataactgctggccatagtctgtttacagttagaaatggagaaataaaggaagtttctggagatgggataaaagaaggtgaccttattgtagcaccaaagaaaattaaactcaatgaaaaaggggtaagcataaacattcccgagttaatctcagatctttccgaggaagaaacagccgacattgtgatgacgatttcagccaagggcagaaagaacttctttaaaggaatgctgagaactttaaggtggatgtttggagaagaaaatagaaggataagaacatttaatcgctatttgttccatctcgaaaaactaggccttatcaaactactgccccgcggatatgaagttactgactgggagagattaaagaaatataaacaactttacgagaagcttgctggaagcgttaagtacaacggaaacaagagagagtatttagtaatgttcaacgagatcaaggattttatatcttacttcccacaaaaagagctcgaagaatggaaaattggaactctcaatggctttagaacgaattgtattctcaaagtcgatgaggattttgggaagctcctaggttactatgttagtgagggctatgcaggtgcacaaaaaaataaaactggtggtatcagttattcggtgaagctttacaatgaggaccctaatgttcttgagagcatgaaaaatgttgcagaaaaattctttggcaaggttagagttgacagaaattgcgtaagtatatcaaagaagatggcatacttagttatgaaatgcctctgtggagcattagccgaaaacaagagaattccttctgttatactcacctctcccgaaccggtacggtggtcatttttagaggcgtattttacaggcgatggagatatacatccatcaaaaaggtttaggctctcaacaaaaagcgagctccttgcaaatcagcttgtgttcttgctgaactctttgggaatatcctctgtaaagataggctttgacagtggggtctatagagtgtatataaatgaagacctgcaatttccacaaacgtctagggagaaaaacacatactactctaacttaattcccaaagagatccttagggacgtgtttggaaaagagttccaaaagaacatgacgttcaagaaatttaaagagcttgttgactctggaaaacttaacagggagaaagccaagctcttggagttcttcattaatggagatattgtccttgacagagtcaaaagtgttaaagaaaaggactatgaagggtatgtctatgacctaagcgttgaggataacgagaactttcttgttggttttggtttgctctatgctcacaacagctattacggctatatggggtatcctaaggcaagatggtactcgaaggaatgtgctgaaagcgttaccgcatgggggagacactacatagagatgacgataagagaaatagaggaaaagttcggctttaaggttctttatgcggacagtgtctcaggagaaagtgagatcataataaggcaaaacggaaagattagatttgtgaaaataaaggatcttttctctaaggtggactacagcattggcgaaaaagaatactgcattctcgaaggtgttgaagcactaactctggacgatgacggaaagcttgtctggaagcccgtcccctacgtgatgaggcacagagcgaataaaagaatgttccgcatctggctgaccaacagctggtatatagatgttactgaggatcattctctcataggctatctaaacacgtcaaaaacgaaaactgccaaaaaaatcggggaaagactaaaggaagtaaagccttttgaattaggcaaagcagtaaaatcgctcatatgcccaaatgcaccgttaaaggatgagaataccaaaactagcgaaatagcagtaaaattctgggagctcgtaggattgattgtaggagatggaaactggggtggagattctcgttgggcagagtattatcttggactttcaacaggcaaagatgcagaagagataaagcaaaaacttctggaacccctaaaaacttatggagtaatctcaaactattacccaaaaaacgagaaaggggacttcaacatcttggcaaagagccttgtaaagtttatgaaaaggcactttaaggacgaaaaaggaagacgaaaaattccagagttcatgtatgagcttccggttacttacatagaggcatttctacgaggactgttttcagctgatggtactgtaactatcaggaagggagttccagagatcaggctaacaaacattgatgctgactttctaagggaagtaaggaagcttctgtggattgttggaatttcaaattcaatatttgctgagactactccaaatcgctacaatggtgtttctactggaacctactcaaagcatctaaggatcaaaaataagtggcgttttgctgaaaggataggctttttaatcgagagaaagcagaagagacttttagaacatttaaaatcagcgagggtaaaaaggaataccatagattttggctttgatcttgtgcatgtgaaaaaagtcgaagagataccatacgagggttacgtttatgacattgaagtcgaagagacgcataggttctttgcaaacaacatcctggtacacaatactgacggcttttatgccacaatacccggggaaaagcctgaactcattaaaaagaaagccaaggaattcctaaactacataaactccaaacttccaggtctgcttgagcttgagtatgagggcttttacttgagaggattctttgttacaaaaaagcgctatgcagtcatagatgaagagggcaggataacaacaaggggcttggaagtagtaaggagagattggagtgagatagctaaggagactcaggcaaaggttttagaggctatacttaaagagggaagtgttgaaaaagctgtagaagttgttagagatgttgtagagaaaatagcaaaatacagggttccacttgaaaagcttgttatccatgagcagattaccagggatttaaaggactacaaagccattggccctcatgtcgcgatagcaaaaagacttgccgcaagagggataaaagtgaaaccgggcacaataataagctatatcgttctcaaagggagcggaaagataagcgatagggtaattttacttacagaatacgatcctagaaaacacaagtacgatccggactactacatagaaaaccaagttttgccggcagtacttaggatactcgaagcgtttggatacagaaaggaggatttaaggtatcaaagctcaaaacaaaccggcttagatgcatggctcaagaggtagctctgttgctttttagtccaagtttctccgcgagtctctctatctctcttttgtattctgctatgtggttttcattcactattaagtagtccgccaaagccataacgcttccaattccaaacttgagctctttccagtctctggcctcaaattcactccatgtttttggatcgtcgcttctccctcttctgctaagcctctcgaatctttttcttggcgaagagtgtacagctatgatgattatctcttcctctggaaacgcatctttaaacgtctgaatttcatctagagacctcactccgtcgattataactgccttgtacttctttagtagttcttttacctttgggatcgttaattttgccacggcattgtccccaagctcctgcctaagctgaatgctcacactgttcataccttcgggagttcttgggatcc

SEQ ID NO: 5 - protein, 15 aa, Thermococcus litoralis
AIKLLANSYYGYMGY

SEQ ID NO: 6 - protein, 15 aa, Pyrococcus Sp. (GB-D)
AIKILANSYYGYYGY

SEQ ID NO: 7 - protein, 15 aa, Thermococcus sp.
AIKILANSFYGYYGY

SEQ ID NO: 8 - protein, 15 aa, Pyrococcus furiosus
AIKLLANSFYGYYGY

SEQ ID NO: 9 - protein, 15 aa, Thermococcus fumicolans
AIKILANSFYGYYGY

SEQ ID NO: 10 - protein, 15 aa, Thermococcus gorgonarius
AIKILANSFYGYYGY

SEQ ID NO: 11 - protein, 15 aa, Thermococcus sp. (TY)
AVKLLANSYYGYMGY

SEQ ID NO: 12 - protein, 15 aa, Pyrococcus abyssi
AIKILANSYYGYYGY

SEQ ID NO: 13 - protein, 15 aa, Pyrococcus glycovaorans
AIKILANSYYGYYGY

SEQ ID NO: 14 - protein, 15 aa, Pyrococcus horikoshii
AIKILANSYYGYYGY

SEQ ID NO: 15 - protein, 15 aa, Pyrococcus sp. (GE23)
AIKILANSYYGYYGY

SEQ ID NO: 16 - protein, 15 aa, Pyrococcus Sp. (KOD1)
AIKILANSYYGYYGY

SEQ ID NO: 17 - protein, 15 aa, Pyrococcus woesei
AIKLLANSFYGYYGY

SEQ ID NO: 18 - protein, 15 aa, Archaeoglobus fulgidus
TLKVLTNSFYGYMGW

SEQ ID NO: 19 - protein, 15 aa, Cenarchaeum symbiosum
ALKVVLNASYGVMGA

SEQ ID NO: 20 - protein, 15 aa, Methanococcus jannaschii
SIKILANSVYGYLAF

SEQ ID NO: 21 - protein, 15 aa, Methanococcus voltae
SIKVLANSHYGYLAF

SEQ ID NO: 22 - protein, 15 aa, Pyrodictium occultum
ALKVLANASYGYMGW

SEQ ID NO: 23 - protein, 15 aa, Sulfurisphaera ohwakuensis
AMKVFINATYGVFGA

SEQ ID NO: 24 - protein, 15 aa, Sulfolobus acidocaldarius
AMKVFINATYGVFGA

SEQ ID NO: 25 - protein, 15 aa, Sulfolobus solfataricus
AMKVFINATYGVFGA

SEQ ID NO: 26 - protein, 15 aa, Herpesvirus
AIKVVCNSVYGFTGV

SEQ ID NO: 27 - protein, 15 aa, human herpesvirus 2
AIKVVCNSVYGFTGV

SEQ ID NO: 28 - protein, 15 aa, Human cytomegalovirus
ALKVTCNAFYGFTGV

SEQ ID NO: 29 - protein, 15 aa, Human DNA Polymerase alpha
ALKLTANSMYGCLGF

SEQ ID NO: 30 - protein, 15 aa, Phage T4
NRKILINSLYGALGN

SEQ ID NO: 31 - DNA, 635 bp, Consensus using 9 degrees N or AmpliTaq
  misc_feature (2)..(3): n is a, c, g, or t
tnntsggaaanncnggcsattgccaatsttgcatkcctkcaggtcngactctagaggatcccygggtaccgagctcngaattcngtaatcatggtcatagctgtttccttgtgtgaaattgttatccngctcacaattccacacaacatacngagccggaagcataaagtgtaaagcctggggtgcctaatgagtgagctaactcacattaattgcgttgcgctcacttgcccgctttccagtcgggaaacctgtcgtgccagctgcattaatgaatcggccggagaggcggtttgcgtattgggcgccagggtggtttttcttttcaccagtgagacgggcaacagctgattgcccttcaccgcctggccytgagagagttgcagcaagcggtccacgctggtttgccccagcaggcgaaaatatggtggttccgaaatcggcaaaatcccttataaatcaaaagaatagccccgagatagggttgaagtgttgttccagtttggaacaagagtccmctattaaagaamgtggactccaacgkcaaagggcgaaaaaccgtctwtcaggggcgatggccactacgkkaaccwtcmcctaatcaagtttttggggkcgagkggccgttaagccta

SEQ ID NO: 32 - DNA, 615 bp, M13mp18 bacteria phage DNA
  misc_feature (2)..(3): n is a, c, g, or t
tnntcnacggccattgccaancttgcatgcctgcaggtcgactctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccggagaggcggtttgcgtattgggcgccagggtggtttttcttttcaccagtgagacgggcaacagctgattgcccttcaccgcctggccctgagagagttgcagcaagcggtccacactggtttgccccagcaggcgaaaatatggtggttccgaaatcggcaaaatcccttataaatcaaaagaatagcccgagatanggttgaagtgttgttccagtttggaacaagagtccactattaaagaaagtggactccaacgtcnaanggcgaaaaaccgtctatcaggggcnatggccactacgttaancatcaccaatcaatttttggggtcagtgcctaagccta

SEQ ID NO: 33 - DNA, 602 bp, M13mp18 bacteria phage DNA
  misc_feature (8)..(9): n is a, c, g, or t
tgggaaanncnggcgagccnatgttnnattncttnaggcngctctngaggatccctgggnccggctcngaattcngtaatcatggtcatagctgtttccttgtgtgaaattgttatccngctcncaattccncacaacntacngagccggaagctaaagtgtaaagctggggnnctaatgagtgagctaactcncnttaattgcgttgcntcncttgccngtttccagtcgggaaactgtcgtgcngctgcnttaatgaatcggccggagaggcggtttgcgtattgggcgcagggnggtttttcttttcaccagtgagacgggcnacngtgattgccttcnccgctgnccttgagagagttgcngcnagcggtcccntggtttgcccagcagggaaaatatggtggtccgaaatcggnaaatccttntaaatcnaaagaatagccccgagatngggttgagtgttgtccagtttggaacaagagcccctattaaagaacgnggactccaacggcaaagggcgaaaaaccgctttcngggcgatggccctncgggaaccttcccctaatcnagtttttgggggcgaggggccgttangccta

(19) United States
(12) Patent Application Publication
(10) Pub. No.: US 2006/0199214 A1
(43) Pub. Date: Sep. 7, 2006

(54) Incorporation of modified nucleotides by archaeon DNA polymerases and related methods

(76) Inventors: William E. Jack, Wenham, MA (US); Andrew F. Gardner, Boston, MA (US); Philip R. Buzby, Brockton, MA (US); James J. DiMeo, Needham, MA (US)

Correspondence Address: HARRIET M. STRIMPEL; NEW ENGLAND BIOLABS, INC., 240 COUNTY ROAD, IPSWICH, MA, 01938-2723, US

(21) Appl. No.: 11/417,625

(22) Filed: May 4, 2006

Related U.S. Application Data
(63) Continuation of application No. 10/089,027, filed on Mar. 26, 2002.
(60) Provisional application No. 60/157,204, filed on Sep. 30, 1999.

(51) Int. Cl. C12Q 1/68; C07H 21/04; C12P 19/34; C12N 9/22; C12N 1/21; C12N 15/74

(52) U.S. Cl. 435/6; 435/91.1; 435/252.3; 435/471; 435/199; 536/23.2

(57) ABSTRACT

The present invention is directed toward improving the efficiency of chain terminator incorporation by Family B archaeon DNA polymerases. Previously, the low efficiency of ddNTP, and more especially dye-labeled ddNTP, incorporation has limited the usefulness of this group of DNA polymerases in protocols requiring chain terminator incorporation.

### CROSS REFERENCE

This application is a continuation of Application Serial No. 10/089,027, which is a §371 of PCT Application No. PCT/US00/26900 filed Sept. 29, 2000, which claims priority to Provisional Application Ser. No. 60/157,204 filed Sept. 30, 1999.

### BACKGROUND OF THE INVENTION

DNA polymerases have played a central role in the development of molecular biology. Their use is at the core of a wide range of laboratory protocols, including DNA sequencing (Sanger, et al., Proc. Natl. Acad. Sci., USA 74:5463-5467 (1977)), strand displacement amplification (SDA; Walker, et al., Proc. Natl. Acad. Sci., USA 89:392-396 (1992)), probe labeling, site-directed mutagenesis, the polymerase chain reaction (PCR; Saiki, et aL, Science, 230:1350-1354 (1985)), and cloning. These applications depend critically on the ability of polymerases to faithfully replicate DNA, either to create a product whose biological properties are identical to the substrate, or to create a product whose identity accurately reflects the substrate, thus facilitating characterization and manipulation of this substrate.

A number of applications require polymerases that are able to incorporate modified nucleotides. One such application is chain terminator nucleic acid sequencing where nucleotides with modified sugars, most often a dideoxynucleotide (ddNTP), are employed to deduce the ordering of bases in a sequencing sample (Sanger, et al., supra. (1977)). Sequence-specific chain termination, occurring upon incorporation of these analogs, creates a product whose length measures the position of the complementary base in the substrate molecule. By ordering such products derived using terminators corresponding to A, G, C and T bases, the nucleic acid sequence can be deduced. Production of these terminator products can occur in four parallel reactions, each with a single A, G, C or T terminator, to deduce the DNA sequence. Alternatively, if each of the four terminators contains a unique detection agent, the terminator products can be produced simultaneously in one reaction. One family of such detection agents is fluorescent probes (Prober, et al., Science 238:336-341 (1987); U.S. Pat. No. 5,332,666). Fluorescent probes can either be used individually or, if the emission spectra are distinguishable, in multiple sets. Fluorescent probes are most often attached to the nucleotide base, creating a need in the art for DNA polymerases that can readily incorporate such dye-labeled nucleotides. Detection probes can also be moieties that interact with a second molecule, such as an antibody, with indirect detection occurring via the second molecule. Such is the case, for example, with the binding of specific antibodies to fluorescein or the binding of streptavidin to biotin. Finally, detection probes can also be radioisotopes, detectable by such methods as autoradiography.

Modifications of chain terminator DNA sequencing have been described that focus on single nucleotide loci, allowing detection of single nucleotide polymorphisms (SNPs; Sarkar, et al., Genomics 13:441-443 (1992); Nikiforov, etal., Nucleic Acids Res. 22:4167-4175; Chen and Kwok, Nucleic Acids Res. 15:347-353 (1997); Chen, et aL, Genome Research 9:492-498 (1999); U.S. Pat. Nos. 5,888,819, 5,952,174, 6,004,744, and 6,013,43). Such single base detection methods have been instructive in genetic testing and analysis, and also require DNA polymerases able to incorporate modified nucleotides with attached detection probes and/or chain terminating functionalities.

A difficulty with methods requiring the incorporation of modified nucleotides is the inherent fidelity of DNA polymerases. As might be expected, incorporation of a number of nucleotide analogs by DNA polymerases is less efficient than incorporation of the naturally occurring residues, namely dATP, dCTP, dGTP and TTP. As a consequence, technologies relying on incorporation of the analogs can suffer from incomplete and non-uniform incorporation. Accordingly, there is a need in the art for DNA polymerases and nucleotide analog combinations that allow for ready incorporation while retaining base specificity. Since a number of methods require a step in which the DNA is denatured at high temperatures, there is a need for such enzymes that are additionally thermostable.

Several approaches can be envisioned to enhance incorporation of modified nucleotides in vitro. First, use of DNA polymerases that more readily incorporate modified nucleotides. Second, use of DNA polymerase variants that more readily incorporate modified nucleotides. Third, use of nucleotide analogs that are more readily incorporated by the target DNA polymerase. Fourth, use of high concentrations of modified nucleotides, driving incorporation by the law of mass action. This final approach is limited by the requirement for larger quantities of reagent, and from the higher detection background introduced by the unincorporated nucleotide, and thus is not the preferred approach.

Although in theory any DNA polymerase could be used to incorporate modified nucleotides, polymerases derived from different sources can have different spectra of nucleotide and nucleotide analog incorporation efficiencies. Thus, the choice of polymerase is important in analog incorporation. Primary amino acid sequence similarities allow the classification of most DNA polymerases into three Families, A, B and C, according to similarities with Escherichia colipolymerases I, II and III, respectively (Ito and Braithwaite, Nucleic Acids Res., 19:4045-4057 (1991); Heringa and Argos, The Evolutionary Biology of Viruses, Morse, S. S., ed., pp. 87-103, Raven Press, N.Y. (1992)). DNA polymerases of Family A have been the predominant enzymes used in DNA sequence determination, and thus have been most extensively studied with regards to their ability to incorporate chain terminators and dye-labeled nucleotides.

A comparison of two Family A DNA polymerases, the Klenow fragment of DNA polymerase I and T7 DNA polymerase, revealed a remarkable difference in incorporation of chain terminating dideoxynucleoside triphosphate (ddNTPs) (Tabor and Richardson, Proc. Natl. Acad. Sci., USA 92:6339-43 (1995)). Further work established that replacement of F762 in Klenow by tyrosine, the residue present in the analogous position in T7 DNA polymerase, drastically increased the efficiency of ddNTP utilization by that enzyme (Tabor and Richardson, supra.). Replacement of the analogous residue of the thermostable Family A Taq DNA polymerase (F667) gave a similar increase in incorporation efficiency of ddNTPs (U.S. Pat. No. 5,614,365). These examples illustrate both the use of alternate polymerases and of polymerase variants to increase the efficiency of terminator incorporation.

DNA polymerases of other families could also be considered for incorporation of modified nucleotides. Since a number of applications involve a heat step for DNA strand denaturation, thermostable enzymes of Family B have been explored as candidates for incorporation of modified nucleotides, including a number derived from thermophilic archaea. Such enzymes include, but are not limited to, Vent® DNA polymerase, originally isolated from Thermococcus litoralis (Perler, et al, Proc. Natl. Acad. Sci. USA 89:5577- 5581 (1992); U.S. Pat. Nos. 5,500,363, 5,834,285, 5,352,778); Pyrococcus furiosus (Pfu) DNA polymerase (U.S. Pat. Nos. 5,489,523, 5,827,716), Deep Vent® DNA polymerase (U.S. Pat. No. 5,834,285), Thermococcus barossii (Tba) DNA polymerase (U.S. Pat. No. 5,882,904) and 9° N. ™ DNA polymerase (Southworth, et al, Proc. Natl. Acad. Sci. USA 93: 5281-5285 (1996) and U.S. Pat. No. 5,756,334).

Early experiments suggested that archaeon DNA polymerases were not promising candidates for applications requiring incorporation of modified nucleotides such as ddNTPs, more particularly those labeled with dyes. Those enzymes for which kinetic information is available, Vent® and Deep Vent®, both have a relatively high Kₘ for nucleotides (Kong, et al, J. Biol. Chem. 268:1965-1975 (1993); New England Biolabs Catalog 1998/1999, p.73), and at least Vent® and Pfu incorporate unsubstituted ddNTP terminators inefficiently (Gardner and Jack, Nucleic Acids Res. 27:2545-2553 (1999); U.S. Pat. No. 5,827,716). Furthermore, reports indicated poor incorporation of dye-substituted ddNTP terminators in DNA sequencing reactions (“CircumVent®: Questions and Answers,” The NEB Transcript, Sept. 1992, p.12-13) and in arrayed-primer extension format reactions involving dye-labeled dideoxy terminators (Tba DNA polymerase, U.S. Pat. No. 5,882,904).

The thermostable archaeon DNA polymerases are not alone in having difficulty incorporating dye-labeled ddNTPs. For example, even though incorporation of ddNTPs is dramatically increased in F667Y versions of Taq DNA polymerase (U.S. Pat. No. 5,614,365; also know by the trade names Thermo Sequenase™ (Amersham Pharmacia Biotech, Piscataway, N.J.) and AmpliTaq® DNA Polymerase, FS (Perkin-Elmer)), dye-terminator incorporation is still characterized by “. . . less uniform peak height patterns when compared to primer chemistry profiles, suggesting that the dyes and/or their linker arms affect enzyme selectivity.” (Brandis, Nucleic Acids Res. 27:1912-1918 (1999)).

Returning to the case of Vent® DNA polymerase, limited information suggests that certain dye-labeled nucleotides can be incorporated. U.S. Pat. No. 5,723,298 claims to have used the infrared dye-labeled IRD40 dATP as a substrate for polymerization by CircumVent® thermostable polymerase, although no quantitative aspects of the polymerization were disclosed. CircumVent® is a trade name referring to Vent® DNA polymerase (exo-), a 3′-5′ exonuclease-deficient form of Vent® DNA polymerase (New England Biolabs, Beverly, Mass.). More recently, llsey and Buzby presented data at the American Society of Biochemistry and Molecular Biology meeting in May of 1999 regarding incorporation of several dye-substituted nucleotides by a variety of DNA polymerases, including Taq and Vent® (exo-) DNA polymerases (Ilsey and Buzby, The FASEB J 13:A1441 (1999)). Incorporation of nine indocyanine and rhodamine dCTP analogs by five polymerases was evaluated. In the case of Vent® (exo-) and Taq DNA polymerases, two dye-substituted dCTP analogs were identified whose incorporation was preferred over dCTP: the indocyanine analog IC3-dCTP and the rhodamine analog R6G-dCTP. It should be noted that these studies used normal deoxyribose sugars, and thus the ability of these analogs to be incorporated as chain terminators was not addressed.

Although ddNTPs are the dominant chain terminators utilized, other analogs have also been explored as chain terminators. The use of acyclo-nucleoside triphosphates (acyclo-NTPs) in chain terminator DNA sequencing by the Klenow fragment of DNA polymerase I and by AMV reverse transcriptase has been discussed (U.S. Pat. No. 5,558,991). Such acyclo derivatives substitute a 2-hydroxyethoxymethyl group for the 2′-deoxyribofuanosyl sugar normally present in dNTPs. Sequencing patterns produced by these two enzymes were found to be virtually identical for use of ddNTPs and acyclo-NTPs. However, approximately ten-fold higher concentrations of the acyclo derivatives were required to produce equivalent patterns, indicating a greater discrimination against those compounds by the two enzymes tested. Thus, at least for these two enzymes, ddNTPs are favored substrates over acyclo-NTPs.

Incorporation of acyclo-NTPs has also been analyzed with Family B DNA polymerases, especially in light of the antiviral activity of selected acyclo derivatives, specially acyclovir. The mode of drug action, in part, is thought to be the preferential incorporation of the chain terminator acyclovir (9-(2- hydroxyethoxymethyl)guanine] triphosphate) by the viral as opposed to the cellular DNA polymerase, human DNA polymerase alpha (Elion, J. Antimicrob. Chemother. 12 Suppl. B:9-17 (1983)). This conclusion is tempered by the observation that inhibition by acycloguanosine has both competitive and non-competitive components. Evidence has been proffered that the Family B herpes virus type 2 (HSV-2) and human cytomegalovirus (HCMV) DNA polymerases have a preference for insertion of acyclo-GTP over ddGTP (Reid, et al., J. Biol. Chem. 263:3898-3904 (1988)). The same report also indicates a strong preference by human DNA polymerase alpha (also a Family B DNA polymerase) for insertion of ddGTP over acyclo-GTP. The contrasting behaviors of these three Family B DNA polymerases, combined with the complex inhibition patterns observed for the viral polymerases, makes a priori predictions difficult for other Family B DNA polymerases as far as the relative incorporation of ddNTPs and acyclo-NTPs are concemed.

### SUMMARY OF THE PRESENT INVENTION

The present invention is directed toward improving the efficiency of chain terminator incorporation by Family B archaeon DNA polymerases. Previously, the low efficiency of ddNTP, and more especially dye-labeled ddNTP, incorporation has limited the usefulness of this group of DNA polymerases in protocols requiring chain terminator incorporation.

Several innovations are exploited in novel combinations in the present invention to overcome previously-noted limitations in chain terminator incorporation. In accordance with the present invention, derivatized ddNTP terminators are identified that are more efficiently incorporated than the corresponding underivatized ddNTPs. Methods are delineated to identify additional compounds of this type. Such compounds offer a marked advantage over previously tested dye-labeled ddNTPs whose incorporation was disfavored.

In certain preferred embodiments, acyclo-NTP terminators are found to be more efficiently incorporated than the corresponding ddNTPs. As with ddNTPs, incorporation of these acyclo-NTPs can be enhanced by specific base adducts.

In other preferred embodiments, incorporation of acyclo-NTPs and of derivatized ddNTPs and acyclo-NTPs is further enhanced by use of DNA polymerase variants.

Each embodiment confers a significant advantage in terminator incorporation, most strongly seen when the various embodiments are combined in a single reaction. In a most preferred embodiment, a variant DNA polymerase is used to incorporate a derivatized acyclo-NTP, using polymerase variants and derivatized terminators typified in the present invention. This novel arrangement provides a vast increase in terminator incorporation over that previously reported.

The efficient production of chain terminator products has obvious application in DNA sequence determination. This arises not only in traditional chain terminator sequencing, but also in automated procedures where detection is via incorporation of dye-labeled terminators. The present invention is applicable to both long range DNA sequence determination where hundreds of base pairs of contiguous sequence are revealed, and to short range sequencing, defining as little as one base pair of sequence. In the case of short range sequencing, the present invention is useful in analyzing sequence polymorphisms, for example in genetic testing and screening for specific single nucleotide polymorphisms (SNPs). Characterization of SNPs can be either by virtue of molecular weight or label incorporation, in either case accommodated by methods described in the present invention.

### BRIEF DESCRIPTION OF THE DRAWINGS

FIG. 1 shows the incorporation of modified ddCTP bases by Vent® (exo-) and Thermo Sequenase™ DNA polymerases. Extension of a [³²P]-labeled primer on an M13mp18 single-stranded substrate was examined in the presence of a 1:1 ratio or 1:10 ratio of analog to dNTP. A reaction containing a 1:1 ratio of unmodified ddCTP to dNTP is used for reference in the first lane. Lanes marked “dNTP” are control reactions performed in the absence of terminators. A, Vent® (exo-). B, Thermo Sequenase™.

FIG. 2 compares dye-labeled ddCTP and dye-labeled acyclo-CTP incorporation by Vent® (exo-) and Thermo Sequenase™ DNA polymerases. Extension of a [³²P]-labeled primer on an M13mp18 single-stranded substrate was examined in the presence of a 1:1 ratio or 1:10 ratio of analog to dNTP. In each panel, a reaction containing a 1:1 ratio of unmodified ddCTP to dNTP is used for reference in the first lane. A, Vent® (exo-). B, Thermo Sequenase™.

FIG. 3 demonstrates the incorporation efficiency of ROX-acyclo-CTP by Vent® (exo-), Deep Vent® (exo-), Pfu (exo-) and 9° N™ (exo-) DNA polymerases. Numbers refer to the ratio of ROX-acyclo-CTP : dCTP in the reaction mixture. The lane labeled “dNTP” illustrates a control reaction not containing terminators.

FIG. 4 shows the incorporation of modified ddCTP bases by Vent® (exo-)/A488L DNA polymerase. Extension of a [³²P]-labeled primer on an M13mp18 single-stranded substrate was examined in the presence of a 1:1 ratio or 1:10 ratio of analog to dNTP. In each panel, a reaction containing a 1:1 ratio of unmodified ddCTP to dNTP is used for reference in the first lane and a reaction containing dNTP but lacking terminators is also shown.

FIG. 5 compares the incorporation efficiency of ROX-ddCTP by Vent® (exo-), Vent® (exo-)/A488L and Vent® (exo-)Y499L DNA polymerases. Numbers refer to the ratio of ROX-ddCTP:dCTP in the reaction mixture. The reaction in the lane labeled “dNTP” contains no chain terminators.

FIG. 6 compares the incorporation efficiency of ROX-ddCTP and ddCTP by Vent® (exo-), Vent® (exo-)/A488L, 9° N™ (exo-) and 9° N™ (exo-)/A485L DNA polymerases. Numbers refer to the ratio of ROX-ddcTP:dcTP or ddCTP:dCTP in the reaction mixture.

FIG. 7 compares incorporation of ROX, IRD700 and TAMRA dye-labeled ddCTP and acyclo-CTP by Vent® (exo-)/A488L DNA polymerase. Numbers refer to the ratio of ROX-ddCTP:dCTP in the reaction mixture.

FIG. 8 compares incorporation of ddCTP and IRD700, ROX and TAMRA dye-labeled acyclo-CTP by Vent® (exo-)/A488L, 9° N™ (exo-)/A485L and Thermo Sequenase™ DNA polymerases. The terminator was present in a 1:1 ratio with dCTP in all cases. Lanes marked dNTP delineate reactions without added terminators.

FIG. 9 compares incorporation of ddGTP and acyclo-GTP by Thermo Sequenase™ and 9° N™ (exo-)/A485L DNA polymerases. Numbers refer to the ratio of terminator:dGTP in the reaction mixture.

FIG. 10 illustrates the output of an ABI377 automated DNA sequencer with samples generated with either 9° N™ (exo-)/A485L DNA Polymerase or AmpliTaq® DNA Polymerase, FS. The DNA sequence along the top line is the consensus sequence from the two unedited traces, while those above the traces are sequences assigned by AutoAssembler software (Perkin-Elmer Corp.).

### DETAILED DESCRIPTION OF THE PRESENT INVENTION

In accordance with the present invention, three innovations are utilized to facilitate incorporation of modified nucleotides by DNA polymerases. Each of these three elements makes a significant individual contribution to the efficiency of incorporation, and can act in concert with the other elements to further enhance incorporation of chain terminators. These elements are, (1) functionalities whose attachment to nucleotide bases can enhance incorporation of that base relative to the naturally-occurring base, and methods to identify such compounds, (2) acyclo-NTPs, based on the discovery that such compounds are more readily incorporated than corresponding ddNTP derivatives by archaeon DNA polymerases and (3) identification and use of archaeon DNA polymerases and polymerase variants with enhanced ability to incorporate nucleotides with modified sugars, specifically chain terminators such as ddNTPs and acyclo-NTPs.

Identifying DNA Polymerases with Similar Properties

As mentioned above, DNA polymerases can be categorized into three families, with enzymes such as Vent® falling into Family B. DNA polymerases within a family can be further subdivided into groups with similar features. Such groupings can be made by several criteria. First, through analytical methods that detect the degree of homology in the underlying nucleic acid sequences encoding the gene. Such similarities are sufficient in many cases to isolate similar genes from alternate organisms, and has been used to discover new archaeon Family B DNA polymerases, as described in U.S. Pat. No. 5,500,363. In that invention, specific DNA probes and hybridization conditions are described to allow for detection by Southern Blot, and isolation of such similar DNA polymerases. The DNA fragment encoding the DNA polymerase was identified as that hybridizes in a Southern blot to an isolated DNA fragment selected from the group consisting of a DNA fragment having nucleotides 1-1274 of SEQ ID NO:4, a DNA fragment having nucleotides 291-1772 of SEQ ID NO:4, a DNA fragment having nucleotides 3387-3533 of SEQ ID NO:4, a DNA fragment having nucleotides 4704-5396 of SEQ ID NO:4, and a DNA fragment having nucleotides 4718-5437 of SEQ ID NO:4, wherein hybridization is conducted under the following conditions: a) hybridization: 0.75 M NaCL, 0.15 M Tris, 10 mM EDTA, 0.1% sodium pyrophosphate, 0.1% sodium lauryl sulfate, 0.03% BSA, 0.03% Ficoll 400, 0.03% PVP and 100 μg/ml boiled calf thymus DNA at 50° C. for about 12 hours and; b) wash: 3×30 minutes with 0.1X SET, 0.1% SDS, 0.1% sodium pyrophosphate and 0.1 M phosphate buffer at 45° C.

SEQ ID NO:4
GAATTCGCGATAAAATCTATTTTCTTCCTCCATTTTTCAATTTCAAAAACGTAAGCATGA 60
GCCAAACCTCTCGCCCTTTCTCTGTCCTTCCCGCTAACCCTCTTGAAAACTCTCTCCAAA 120
GCATTTTTTGATGAAAGCTCACGCTCCTCTATGAGGGTCAGTATATCTGCAATGAGTTCG 180
TGAAGGGTTATTCTGTAGAACAACTCCATGATTTTCGATTTGGATGGGGGTTTAAAAATT 240
TGGCGGAACTTTTATTTAATTTGAACTCCAGTTTATATCTGGTGGTATTTATGATACTGG 300
ACACTGATTACATAACAAAAGATGGCAAGCCTATAATCCGAATTTTTAAGAAAGAGAACG 360
GGGAGTTTAAAATAGAACTTGACCCTCATTTTCAGCCCTATATATATGCTCTTCTCAAAG 420
ATGACTCCGCTATTGAGGAGATAAAGGCAATAAAGGGCGAGAGACATGGAAAAACTGTGA 480
GAGTGCTCGATGCAGTGAAAGTCAGGAAAAAATTTTTGGGAAGGGAAGTTGAAGTCTGGA 540
AGCTCATTTTCGAGCATCCCCAAGACGTTCCAGCTATGCGGGGCAAAATAAGGGAACATC 600
CAGCTGTGGTTGACATTTACGAATATGACATACCCTTTGCCAAGCGTTATCTCATAGACA 660
AGGGCTTGATTCCCATGGAGGGAGACGAGGAGCTTAAGCTCCTTGCCTTTGATATTGAAA 720
CGTTTTATCATGAGGGAGATGAATTTGGAAAGGGCGAGATAATAATGATTAGTTATGCCG 780
ATGAAGAAGAGGCCAGAGTAATCACATGGAAAAATATCGATTTGCCGTATGTCGATGTTG 840
TGTCCAATGAAAGAGAAATGATAAAGCGTTTTGTTCAAGTTGTTAAAGAAAAAGACCCCG 900
ATGTGATAATAACTTACAATGGGGACAATTTTGATTTGCCGTATCTCATAAAACGGGCAG 960
AAAAGCTGGGAGTTCGGCTTGTCTTAGGAAGGGACAAAGAACATCCCGAACCCAAGATTC 1020
AGAGGATGGGTGATAGTTTTGCTGTGGAAATCAAGGGTAGAATCCACTTTGATCTTTTCC 1080
CAGTTGTGCGAAGGACGATAAACCTCCCAACGTATACGCTTGAGGCAGTTTATGAAGCAG 1140
TTTTAGGAAAAACCAAAAGCAAATTAGGAGCAGAGGAAATTGCCGCTATATGGGAAACAG 1200
AAGAAAGCATGAAAAAACTAGCCCAGTACTCAATGGAAGATGCTAGGGCAACGTATGAGC 1260
TCGGGAAGGAATTCTTCCCCATGGAAGCTGAGCTGGCAAAGCTGATAGGTCAAAGTGTAT 1320
GGGACGTCTCGAGATCAAGCACCGGCAACCTCGTGGAGTGGTATCTTTTAAGGGTGGCAT 1380
ACGCGAGGAATGAACTTGCACCGAACAAACCTGATGAGGAAGAGTATAAACGGCGCTTAA 1440
GAACAACTTACCTGGGAGGATATGTAAAAGAGCCAGAAAAAGGTTTGTGGGAAAATATCA 1500
TTTATTTGGATTTCCGCAGTCTGTACCCTTCAATAATAGTTACTCACAACGTATCCCCAG 1560
ATACCCTTGAAAAAGAGGGCTGTAAGAATTACGATGTTGCTCCGATAGTAGGATATAGGT 1620
TCTGCAAGGACTTTCCGGGCTTTATTCCCTCCATACTCGGGGACTTAATTGCAATGAGGC 1680
AAGATATAAAGAAGAAAATGAAATCCACAATTGACCCGATCGAAAAGAAAATGCTCGATT 1740
ATAGGCAAAGGGCTATTAAATTGCTTGCAAACAGCATCTTACCCAACGAGTGGTTACCAA 1800
TAATTGAAAATGGAGAAATAAAATTCGTGAAAATTGGCGAGTTTATAAACTCTTACATGG 1860
AAAAACAGAAGGAAAACGTTAAAACAGTAGAGAATACTGAAGTTCTCGAAGTAAACAACC 1920
TTTTTGCATTCTCATTCAACAAAAAAATCAAAGAAAGTGAAGTCAAAAAAGTCAAAGCCC 1980
TCATAAGACATAAGTATAAAGGGAAAGCTTATGAGATTCAGCTTAGCTCTGGTAGAAAAA 2040
TTAACATAACTGCTGGCCATAGTCTGTTTACAGTTAGAAATGGAGAAATAAAGGAAGTTT 2100
CTGGAGATGGGATAAAAGAAGGTGACCTTATTGTAGCACCAAAGAAAATTAAACTCAATG 2160
AAAAAGGGGTAAGCATAAACATTCCCGAGTTAATCTCAGATCTTTCCGAGGAAGAAACAG 2220
CCGACATTGTGATGACGATTTCAGCCAAGGGCAGAAAGAACTTCTTTAAAGGAATGCTGA 2280
GAACTTTAAGGTGGATGTTTGGAGAAGAAAATAGAAGGATAAGAACATTTAATCGCTATT 2340
TGTTCCATCTCGAAAAACTAGGCCTTATCAAACTACTGCCCCGCGGATATGAAGTTACTG 2400
ACTGGGAGAGATTAAAGAAATATAAACAACTTTACGAGAAGCTTGCTGGAAGCGTTAAGT 2460
ACAACGGAAACAAGAGAGAGTATTTAGTAATGTTCAACGAGATCAAGGATTTTATATCTT 2520
ACTTCCCACAAAAAGAGCTCGAAGAATGGAAAATTGGAACTCTCAATGGCTTTAGAACGA 2580
ATTGTATTCTCAAAGTCGATGAGGATTTTGGGAAGCTCCTAGGTTACTATGTTAGTGAGG 2640
GCTATGCAGGTGCACAAAAAAATAAAACTGGTGGTATCAGTTATTCGGTGAAGCTTTACA 2700
ATGAGGACCCTAATGTTCTTGAGAGCATGAAAAATGTTGCAGAAAAATTCTTTGGCAAGG 2760
TTAGAGTTGACAGAAATTGCGTAAGTATATCAAAGAAGATGGCATACTTAGTTATGAAAT 2820
GCCTCTGTGGAGCATTAGCCGAAAACAAGAGAATTCCTTCTGTTATACTCACCTCTCCCG 2880
AACCGGTACGGTGGTCATTTTTAGAGGCGTATTTTACAGGCGATGGAGATATACATCCAT 2940
CAAAAAGGTTTAGGCTCTCAACAAAAAGCGAGCTCCTTGCAAATCAGCTTGTGTTCTTGC 3000
TGAACTCTTTGGGAATATCCTCTGTAAAGATAGGCTTTGACAGTGGGGTCTATAGAGTGT 3060
ATATAAATGAAGACCTGCAATTTCCACAAACGTCTAGGGAGAAAAACACATACTACTCTA 3120
ACTTAATTCCCAAAGAGATCCTTAGGGACGTGTTTGGAAAAGAGTTCCAAAAGAACATGA 3180
CGTTCAAGAAATTTAAAGAGCTTGTTGACTCTGGAAAACTTAACAGGGAGAAAGCCAAGC 3240
TCTTGGAGTTCTTCATTAATGGAGATATTGTCCTTGACAGAGTCAAAAGTGTTAAAGAAA 3300
AGGACTATGAAGGGTATGTCTATGACCTAAGCGTTGAGGATAACGAGAACTTTCTTGTTG 3360
GTTTTGGTTTGCTCTATGCTCACAACAGCTATTACGGCTATATGGGGTATCCTAAGGCAA 3420
GATGGTACTCGAAGGAATGTGCTGAAAGCGTTACCGCATGGGGGAGACACTACATAGAGA 3480
TGACGATAAGAGAAATAGAGGAAAAGTTCGGCTTTAAGGTTCTTTATGCGGACAGTGTCT 3540
CAGGAGAAAGTGAGATCATAATAAGGCAAAACGGAAAGATTAGATTTGTGAAAATAAAGG 3600
ATCTTTTCTCTAAGGTGGACTACAGCATTGGCGAAAAAGAATACTGCATTCTCGAAGGTG 3660
TTGAAGCACTAACTCTGGACGATGACGGAAAGCTTGTCTGGAAGCCCGTCCCCTACGTGA 3720
TGAGGCACAGAGCGAATAAAAGAATGTTCCGCATCTGGCTGACCAACAGCTGGTATATAG 3780
ATGTTACTGAGGATCATTCTCTCATAGGCTATCTAAACACGTCAAAAACGAAAACTGCCA 3840
AAAAAATCGGGGAAAGACTAAAGGAAGTAAAGCCTTTTGAATTAGGCAAAGCAGTAAAAT 3900
CGCTCATATGCCCAAATGCACCGTTAAAGGATGAGAATACCAAAACTAGCGAAATAGCAG 3960
TAAAATTCTGGGAGCTCGTAGGATTGATTGTAGGAGATGGAAACTGGGGTGGAGATTCTC 4020
GTTGGGCAGAGTATTATCTTGGACTTTCAACAGGCAAAGATGCAGAAGAGATAAAGCAAA 4080
AACTTCTGGAACCCCTAAAAACTTATGGAGTAATCTCAAACTATTACCCAAAAAACGAGA 4140
AAGGGGACTTCAACATCTTGGCAAAGAGCCTTGTAAAGTTTATGAAAAGGCACTTTAAGG 4200
ACGAAAAAGGAAGACGAAAAATTCCAGAGTTCATGTATGAGCTTCCGGTTACTTACATAG 4260
AGGCATTTCTACGAGGACTGTTTTCAGCTGATGGTACTGTAACTATCAGGAAGGGAGTTC 4320
CAGAGATCAGGCTAACAAACATTGATGCTGACTTTCTAAGGGAAGTAAGGAAGCTTCTGT 4380
GGATTGTTGGAATTTCAAATTCAATATTTGCTGAGACTACTCCAAATCGCTACAATGGTG 4440
TTTCTACTGGAACCTACTCAAAGCATCTAAGGATCAAAAATAAGTGGCGTTTTGCTGAAA 4500
GGATAGGCTTTTTAATCGAGAGAAAGCAGAAGAGACTTTTAGAACATTTAAAATCAGCGA 4560
GGGTAAAAAGGAATACCATAGATTTTGGCTTTGATCTTGTGCATGTGAAAAAAGTCGAAG 4620
AGATACCATACGAGGGTTACGTTTATGACATTGAAGTCGAAGAGACGCATAGGTTCTTTG 4680
CAAACAACATCCTGGTACACAATACTGACGGCTTTTATGCCACAATACCCGGGGAAAAGC 4740
CTGAACTCATTAAAAAGAAAGCCAAGGAATTCCTAAACTACATAAACTCCAAACTTCCAG 4800
GTCTGCTTGAGCTTGAGTATGAGGGCTTTTACTTGAGAGGATTCTTTGTTACAAAAAAGC 4860
GCTATGCAGTCATAGATGAAGAGGGCAGGATAACAACAAGGGGCTTGGAAGTAGTAAGGA 4920
GAGATTGGAGTGAGATAGCTAAGGAGACTCAGGCAAAGGTTTTAGAGGCTATACTTAAAG 4980
AGGGAAGTGTTGAAAAAGCTGTAGAAGTTGTTAGAGATGTTGTAGAGAAAATAGCAAAAT 5040
ACAGGGTTCCACTTGAAAAGCTTGTTATCCATGAGCAGATTACCAGGGATTTAAAGGACT 5100
ACAAAGCCATTGGCCCTCATGTCGCGATAGCAAAAAGACTTGCCGCAAGAGGGATAAAAG 5160
TGAAACCGGGCACAATAATAAGCTATATCGTTCTCAAAGGGAGCGGAAAGATAAGCGATA 5220
GGGTAATTTTACTTACAGAATACGATCCTAGAAAACACAAGTACGATCCGGACTACTACA 5280
TAGAAAACCAAGTTTTGCCGGCAGTACTTAGGATACTCGAAGCGTTTGGATACAGAAAGG 5340
AGGATTTAAGGTATCAAAGCTCAAAACAAACCGGCTTAGATGCATGGCTCAAGAGGTAGC 5400
TCTGTTGCTTTTTAGTCCAAGTTTCTCCGCGAGTCTCTCTATCTCTCTTTTGTATTCTGC 5460
TATGTGGTTTTCATTCACTATTAAGTAGTCCGCCAAAGCCATAACGCTTCCAATTCCAAA 5520
CTTGAGCTCTTTCCAGTCTCTGGCCTCAAATTCACTCCATGTTTTTGGATCGTCGCTTCT 5580
CCCTCTTCTGCTAAGCCTCTCGAATCTTTTTCTTGGCGAAGAGTGTACAGCTATGATGAT 5640
TATCTCTTCCTCTGGAAACGCATCTTTAAACGTCTGAATTTCATCTAGAGACCTCACTCC 5700
GTCGATTATAACTGCCTTGTACTTCTTTAGTAGTTCTTTTACCTTTGGGATCGTTAATTT 5760
TGCCACGGCATTGTCCCCAAGCTCCTGCCTAAGCTGAATGCTCACACTGTTCATACCTTC 5820
GGGAGTTCTTGGGATCC 5837

In a similar vein, analytical methods can also be used to discover and identify proteins with similar amino acid sequences, for example by using antibodies raised to a first DNA polymerase to identify other related proteins based on cross-reactivity (U.S. Pat. No. 5,500,363).

A second method of grouping is by the degree of identity and/or similarity between the primary amino acid sequence of the polymerases, which the worker skilled in the art will recognize as also being correlated to the underlying gene coding sequence. This method of analysis relies on sequence alignments rather than physical characterization. Several computer programs have been devised to make this comparison between proteins, one of which is BLAST (Altschul, et al. Nucleic Acids Res. 25:3389-3402 (1997); Tatusova, et aL, FEMS Microbiol Lett. 174:247-250 (1999)). Alignments obtained with such programs typically report the percentage of sequence identity and of sequence similarity between two test sequences, although other statistical measures of identity and similarity are also available and can be relied upon. In addition to this global measure of sequence similarity, proteins can also display sequence similarity over short stretches of primary amino acid sequence. While not wishing to be bound by theory, these patches of similarity are thought to occur most often at essential protein interfaces, such as those involved in catalysis, substrate binding or protein- protein recognition. As such, the degree of sequence similarity, particularly in conserved sequence motifs, is predictive of the degree to which the proteins will behave similarly in both physical properties and catalytic function. For example, mutation of the motif associated with 3′-5′ exonuclease activity of DNA polymerases (Bernad, et al., Cell 59:219-228 (1989)) has been shown to abolish this activity in a variety of polymerases (Derbyshire, et al., Science 240:199-201 (1988)). Example 3 illustrates BLAST-derived sequence identity information for selected archaeon DNA polymerases.

Finally, groupings can be defined by functional similarity, assessed by biochemical assays of such features as kinetic parameters (e.g., Kₘ and turnover number), propensity to insert modified nucleotides, template specificity, and sensitivity to changes in reaction conditions such as pH, temperature, salt types and composition, and cofactors (e.g., Mg²⁺). For the purposes of this invention, the most important grouping is by functional assays of the polymerases, i.e. the ability to efficiently incorporate the modified bases described herein.

As the examples will show, the above groupings are inter-related. Thus, DNA polymerases grouped together by sequence similarities, both nucleic acid and amino acid, also tend to have similar biochemical characteristics. Thus, a reasonable prediction is that DNA polymerases showing a greater degree of similarity to those archaeon DNA polymerases in the examples will be most likely to function in the invention described herein.

DNA Polymerase Variants with Diminished Exonuclease Activity

Two general classes of archaeon DNA polymerase variants are utilized in the present invention. First, exonuclease-deficient (exo-) variants. A number of DNA polymerases possess a 3-5′ exonuclease activity, including the Family B DNA polymerases identified in archaea. One function of this activity is “proofreading,” wherein the polymerase can remove 3′ nucleotides before proceeding with polymerization. Incorrectly base-paired, or aberrant nucleotides are preferentially removed by this activity, increasing the fidelity of replication (Kornberg, DNA Replication, W. H. Freeman and Company, San Francisco, p.127 (1980)). While not wishing to be bound by theory, modified nucleotides might reasonably be expected to sensed as aberrant, and, even if incorporated, be subject to removal by this activity. To avoid this possibility, variants have been created that lack or have diminished exonuclease activity (Vent® DNA polymerase: Kong, et al., supra (1993); U.S. Pat. No. 5,352,778; Pyrococcus furiosus (Pfu) DNA polymerase: U.S. Pat. No. 5,489,523; Tba DNA polymerase: U.S. Pat. No. 5,882,904; Deep Vent® DNA polymerase: U.S. Pat. No. 5,834,285, MA; 9° N™ DNA polymerase: Southworth, et al., Proc. Natl. Acad. Sci. USA 93:5281 -5285 (1996); KOD DNA polymerase: U.S. Pat. No. 6,008,025). In each of these cases exonuclease activity was diminished by creating polymerases with specific variations within a common, recognized amino acid sequence motif, enabling the skilled artisan to predict where similar changes could be made in other DNA polymerases to similarly modulate exonuclease activity. One skilled in the art will appreciate the possibility that the exonuclease-deficient forms may not be absolutely required in this application, as suggested in U.S. Pat. No. 5,945,312 for the Family B DNA polymerase derived from bacteriophage T4. The second general class of DNA polymerases variants will be described below.

Incorporation of Dye-Labeled Nucleotides by Archaeon DNA Polymerases

At present, it is difficult to predict on structural or chemical grounds which dye-substituted molecules will be readily incorporated by archaeon DNA polymerases. A limited literature has reported incorporation of nucleotides with dye-labeled bases by archaeon DNA polymerases. U.S. Pat. No. 5,723,298.claims incorporation of IRD40 dATP by Vent®) DNA polymerase, although measures of the efficiency of incorporation were not presented. Recently, Ilsey and Buzby (Ilsey and Buzby, supra.) identified dye-labeled dCTP analogs whose incorporation was enhanced relative to underivatized dCTP by Vent® (exo-) DNA polymerase, as evident in comparative incorporation assays. Ilsey and Buzby noted in the series of compounds tested that the most hydrophobic cyanine dye was the preferred substrate for polymerization (Ilsey and Buzby, supra.). However, due to the limited number of dyes tested, there is little predictive power in this observation.

Incorporation of dye-substituted chain terminators, however, was not examined in the above studies, allowing uncertainty as to whether these results can be extended to instances where the dye is linked to a chain terminator. In fact, initial studies of Vent® (exo-) DNA polymerase suggested that dye-terminator incorporation by Vent® DNA polymerase was not efficient (“CircumVent®: Questions and Answers,”The NEB Transcript, Sept. 1992, p. 12-13). Additionally, U.S. Pat. No. 5,882,904 reports Deep Vent® (exo-) and Tba (exo-) DNA polymerases incorporate FL-ddNTPs many times less efficient than a modified form of Taq DNA polymerase (KlenTaq). Both of these reports leave significant questions as to whether dye-terminators themselves are disfavored, and if alternative dyes or terminator functionalities can influence the observed discrimination against incorporation.

Dye-Terminators that are Efficiently Incorporated by Archaeon DNA Polymerases

In order to determine the extent of dye-terminator incorporation by archaeon DNA polymerases, the titration assay described by Gardner and Jack (supra.) was used (Example 1). In this assay, the efficiency of incorporation of chain terminator nucleotides is judged by the size distribution of reaction products in a polymerization reaction. As the efficiency of chain-terminator incorporation increases, the average reaction product size decreases because polymerization is more often halted by terminator addition. By comparing the amount of terminator required to give the same spectrum of reaction products, the relative efficiency of incorporation of the test compounds with the different polymerases can be determined. Initial determinations compared the ability of Thermo Sequenase™ (Amersham Pharmacia Biotech, Piscataway, N.J.) and Vent® (exo-) (New England Biolabs, Beverly, Mass.) to incorporate a variety of dye-substituted ddCTP analogs, comparing their incorporation to underivatized ddCTP. Dye terminators were obtained from NEN Life Sciences (Boston, Mass.), either as commercial products or as evaluation samples (Table 2).

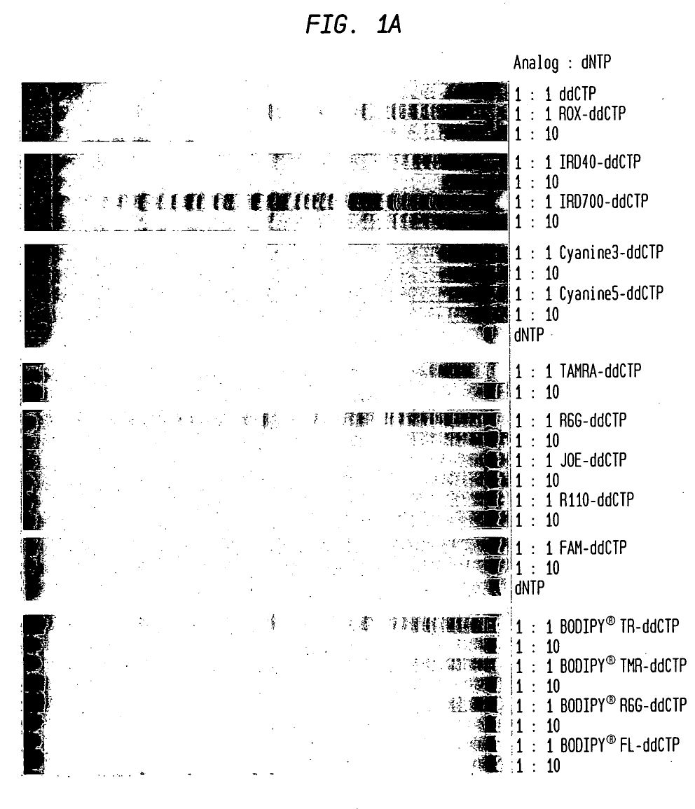

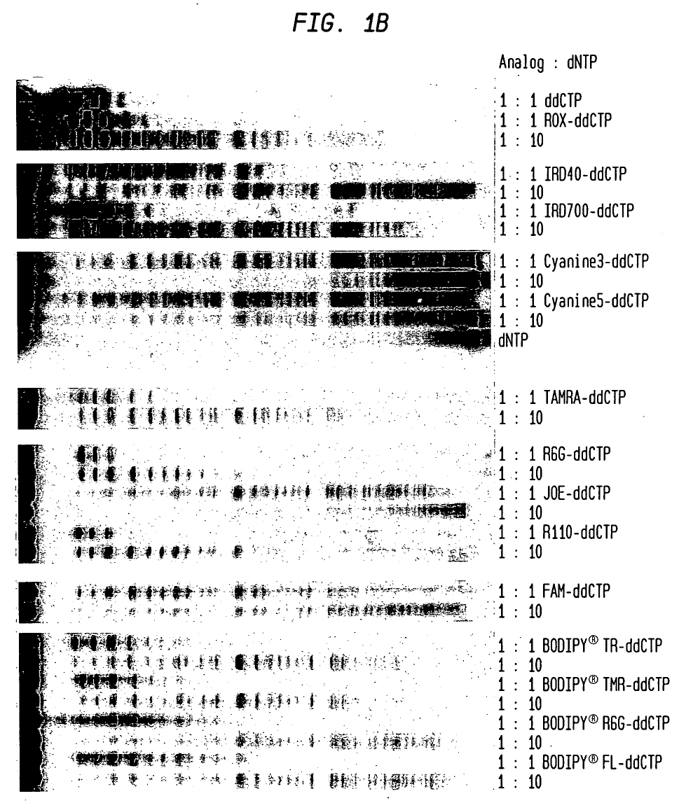

Three classes of dye terminators could be identified with Vent® (exo-) DNA polymerase based on the patterns of termination products produced (Example 2). In the first class banding patterns for ddCTP and the analog had similar concentration dependencies, indicating that the modified base was incorporated no better than the corresponding ddCTP (e.g., IRD40 ddCTP; FIG. 1A). In the second class incorporation of the dye-substituted base was less than that observed with the normal ddCTP as indicated by a dominance of higher molecular weight bands at fixed terminator concentration (e.g., JOE ddCTP; FIG. 1A). In the final class of analogs the distribution of terminated products was shifted to lower molecular weights, indicating an increased incorporation relative to the corresponding ddCTP substrate (e.g., ROX ddCTP, TAMRA ddCTP, BODIPY® TR ddCTP and BODIPY® TMR ddCTP ; FIG. 1A). In this last set of analogs, the presence of the dye enhanced incorporation of the terminator base relative to the parent base ddCTP. Importantly, when appropriate concentrations of analog were compared, a similar band pattern emerged, indicating no loss of base specificity in the insertion of the analogs. Although a limited number of dyes were analyzed by this method, the efficiency of incorporation of alternate dyes could easily be evaluated using the same assay system in conjunction with the desired DNA polymerase. Of course, alternate assays that compare the relative ability of modified nucleotides to be incorporated could also be employed.

Relative Incorporation of Dye-Labeled ddNTP and acyclo-NTP Derivatives

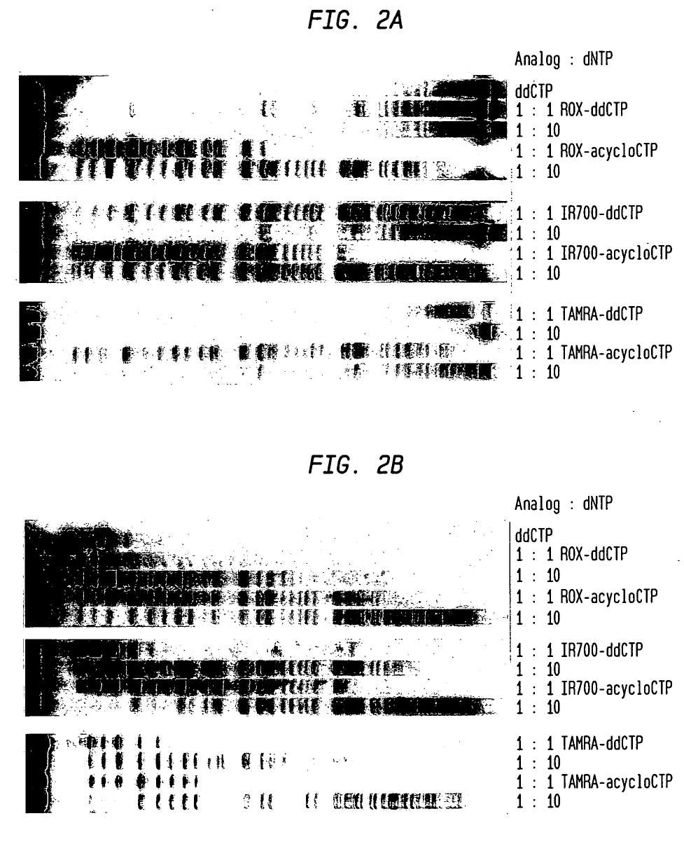

The above experiments identified dyes that, when coupled to ddNTPs, enhanced nucleotide incorporation. Acyclo-NTP derivatives have been used as terminators, although the literature does not report instances of their use with archaeon DNA polymerases. To test whether this alternate terminator might also function in this system, incorporation of dye-acyclo-CTP derivatives were tested using a titration assay (Example 5). In each case tested, acyclo derivatives were more efficiently incorporated by Vent® (exo-) DNA polymerase than those of the corresponding ddNTP (ROX-acyclo-CTP, IRD700-acyclo-CTP and TAMRA-acyclo-CTP; FIG. 2A), indicating that the acyclo chain terminator was even more effectively incorporated than the dideoxy analog. The hierarchy of which dye terminators were more efficiently incorporated was identical for ddNTP and acyclo-NTP derivatives, serving as additional confirmation that the acyclo-NTPs are better incorporated than ddNTPs by the archaeon DNA polymerases (see also Example 12). In contrast, incorporation of acyclo derivatives by Thermo Sequenase™ did not exceeded ddNTP incorporation levels, and in most cases was significantly lower (FIG. 2B).

The similarity in amino acid sequence of Family B archaeon DNA polymerases suggests that these enzymes might share similar dye-terminator incorporation properties. Table 3 is a sampling of other Family B

DNA polymerases from sequence databases, comparing the primary amino acid sequence similarity for the proteins. As described above, those with greatest similarity to the tested enzymes are considered most likely to share the described incorporation properties.

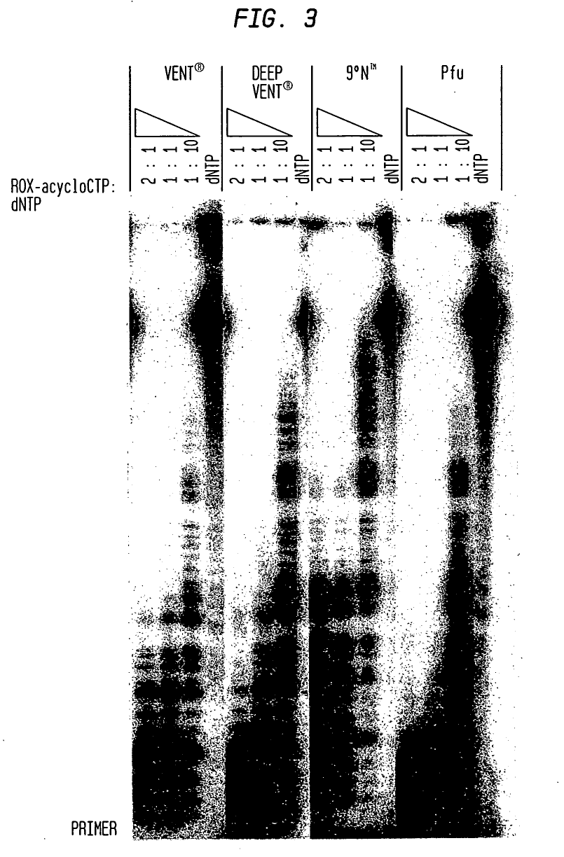

Accordingly, terminator titrations assays were repeated using an expanded set of archaeon DNA polymerases, specifically Vent® (exo-) (New England Biolabs, Beverly, Mass.), Deep Vent® (exo-) (New England Biolabs, Beverly, Mass.), Pfu (exo-) (Stratagene, La Jolla, Calif.) and 9N™ (exo-) (Example 4). The pattern of incorporation preference was identical for these four enzymes (see Example 6, FIG. 3), each demonstrating more efficient incorporation of dye-acyclo-NTPs than the corresponding dye-ddNTPs. Thus, the dye-terminator incorporation properties of one enzyme should be predictive of the incorporation properties of other members of this set.

The similarity of incorporation patterns with these selected enzymes suggests that not only these archaeon DNA polymerases, but a larger family of DNA polymerases could share the ability to incorporate acyclo to a greater extent than dideoxy terminators. Since Pfu, Deep Vent® and 9° N™ DNA polymerases have greater than about 70% sequence identity with Vent DNA polymerase, other enzymes with equivalent or greater identity can reasonably be expected to perform as Vent® (exo-) DNA polymerase in this invention. Notably, those enzymes for which no significant sequence similarity is found (i.e., Family A DNA polymerases such as Taq) do not perform in similar ways in the current invention. This fact leads us to believe that archaeon enzymes showing intermediate identity, namely those between about 20 and 70% identity, are reasonable candidates for this invention. Indeed, the Family B herpes simplex virus type 2 and human cytomegalovirus DNA polymerases in that range of sequence identity have been reported to.incorporate acyclo-GTP to a greater extent than ddGTP (Reid, et al., J. Biol. Chem. 263:3898-3904 (1988)). Some caution must be employed in this regard as the same report indicates human DNA polymerase alpha (27% sequence identity) incorporates ddGTP to a much greater extent than acyclo-GTP (Reid, et al., supra). While not wishing to be bound by theory, we anticipate the lack of utility of the human DNA polymerase alpha in this invention arises from the evolutionary distance [symbol] [symbol] between humans and archaea, and thus should not discourage the worker skilled in the art from screening for the desired activity, for example by using the dye-terminator titrations described in this invention. We additionally note that the experiments by Reid, et al. did not encompass incorporation of dye- labeled substrates.

Vent DNA Polymerase Variants can Increase Dye-Terminator Incorporation

Archaeon DNA polymerase variants have been described that increase the incorporation efficiency for specific classes of chain terminators, namely ddNTPs and 3′-dNTPs. Variants of this type have been described in Vent® DNA polymerase (Gardner and Jack, supra (1999), Pfu DNA polymerase (U.S. Pat. No. 5,827,716) and Tba DNA polymerase (U.S. Pat. No. 5,882,904). In each of these examples, changes within a limited region of the protein primary sequence were made (Table 1), more specifically in Motif B (Delarue, et al., Protein Eng. 3:461-467 (1990)). Most of these changes resulted in a modest (less than 5-fold) increase in incorporation of ddNTPs. Alterations corresponding to A488L in Vent® DNA polymerase produced the greatest increase (approximately 12-fold; Gardner and Jack, supra.), with lesser effects seen with a Y499L variant. It may be noted that the amino acid residues mutated in the above studies are absolutely conserved among the four polymerases tested, as well as among other archaeon DNA polymerases found in data bases (Table 3). It thus seems likely that similar mutations in related DNA polymerases will have similar effects.

TABLE 1
Primary sequence alignment for several
archaeon DNA polymerases

| Vent ® | 9° N ™ | Tba | Pfu |
|---|---|---|---|
| A488 | A485 | A485 | A486 |
| I489 | I486 | I486 | I487 |
| K490 | K487 | K487 | K488 |
| L491 | I488 | I488 | L489 |
| L492 | L489 | L489 | L490 |
| A493 | A490 | A490 | A491 |
| Y499 | Y496 | Y496 | Y497 |

Rows present equivalent amino acid positions in the four DNA polymerases:Thermococcus litoralis (Vent®), Thermococcus sp. 9° N-7 (9° N™) and Thermococcus barossii(Tba). U.S. Pat. No. 5,827,716 discloses modification of residue A491 of Pfu DNA polymerase. U.S. Pat. No. 5,882,904 discloses modification of residue L489 of Tba DNA polymerase.

However, these studies did not address the effects of the cited mutations on dye-terminator incorporation, nor did they address incorporation of alternate terminators such as acyclo-NTPs. There was no teaching to suggest that the increased incorporation of ddNTPs by these variants could overcome the observed difficulty of incorporating dye-labeled ddNTPs (“CircumVent®:Questions and Answers,” supra, U.S. Pat. No. 5,882,904), nor was there a teaching that dye-terminators could be incorporated by archaeon DNA polymerases more efficiently than the corresponding ddNTP. This invention goes beyond those previous studies in identifying dye-terminators better incorporated by archaeon Family B DNA polymerases, and additionally in using DNA polymerase variants to enhance incorporation.

The effects of the A488L mutation in dye-terminator incorporation was tested using the titration assay, comparing Vent® (exo-)/A488L and Vent® (exo-) DNA polymerases (Example 7; compare FIG. 4 to FIGS. 1 and 3). Terminator incorporation was increased in the (exo-)/A488L variant when compared to the (exo-) polymerase. This increase was observed irrespective of whether the terminator was ddNTP or acyclo-NTP, and was observed with all dye-labels analyzed. When considered separately, the preference for acyclo-NTPs as opposed to ddNTPs was preserved, as was the relative preference for specific dyes noted above. Consequently, the increased incorporation reflects effects above and beyond those reported in the previous examples.

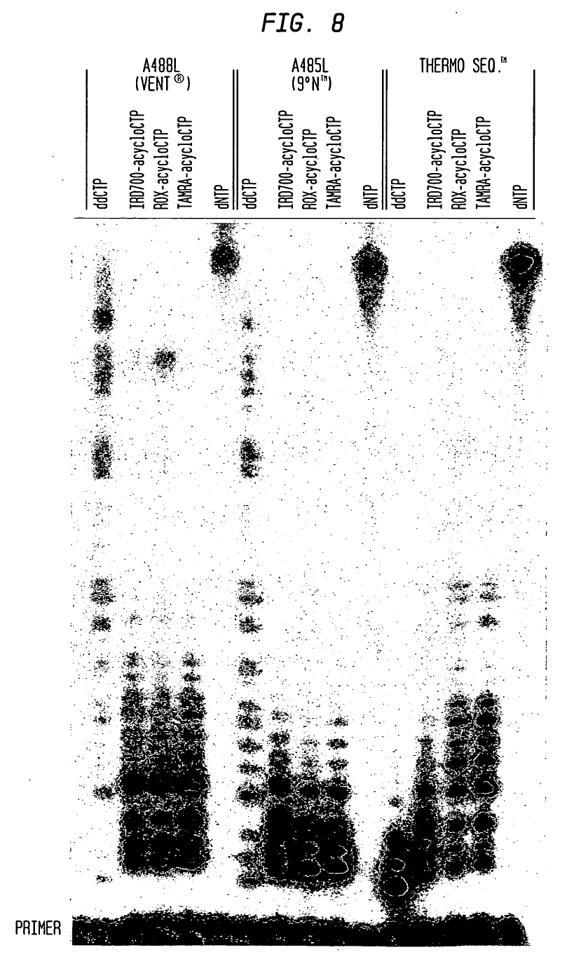

To broaden the applicability of this observation, the analogous 9° N ™ (exo-) DNA polymerase variant A485L (Table 1) was created (Example 9) and employed in the same assays (see Examples 11 and 12). Relative incorporation of dye-terminators was similar to those observed with the Vent® (exo-)/A488L DNA polymerase both with regards to terminator type (i.e., ddNTP or acyclo-NTP; FIGS. 6, 8, and 9) and dye label (FIGS. 6 and 8), establishing the common effects of mutants at this locus among archaeon DNA polymerases.

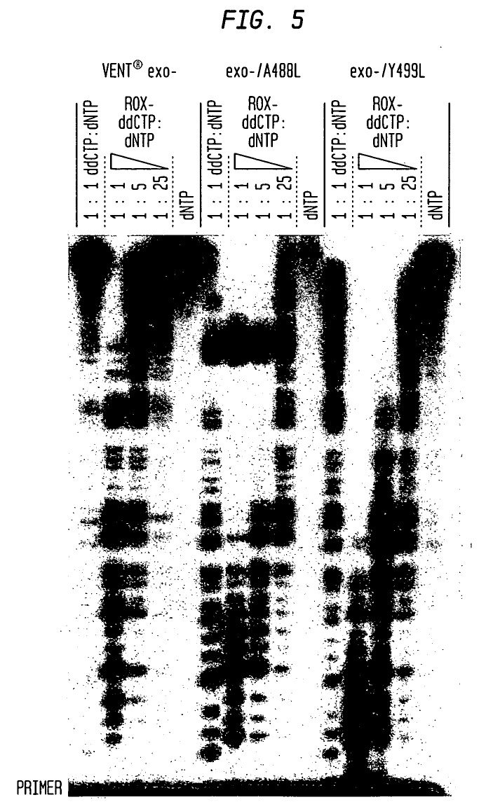

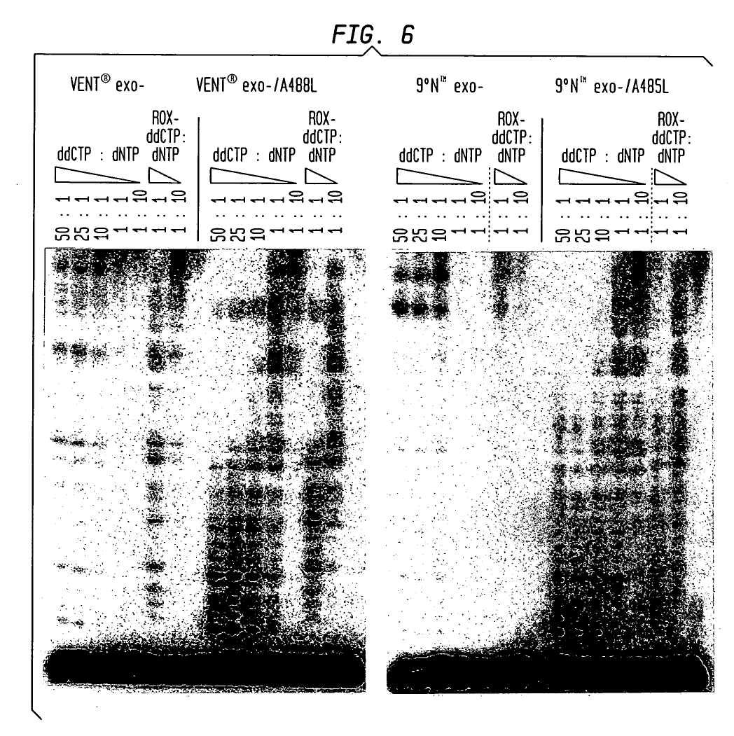

Additional experiments with a second Vent® DNA polymerase variant, (exo-)Y499L, displayed increased incorporation similar in nature to the (exo-)/A488L variant, demonstrating that increased incorporation was not limited to the (exo-)/A488L variant (Example 8; FIG. 5). The worker skilled in the art will appreciate that variants analogous to those noted above in other archaeon DNA polymerases would be predicted to similarly enhance incorporation of comparable modified nucleotides. Additionally, other variants that facilitate incorporation of nucleotides with modified sugars might also be expected to operate in this invention, including but not necessarily limited to variants in residues corresponding to Vent® DNA polymerase residues A488 to Y499. For example, a variant corresponding to Vent® L492 has been described that increased ddNTP incorporation (U.S. Pat. No. 5,882,904). Titration assays, as described herein, could be used to verify the action of this and other similar variants in terminator incorporation.

Relative Incorporation of ddGTP and acyclo-GTP

The experiments noted above gave very strong indications that the basis for enhanced dye-acyclo over dye-dideoxy terminator incorporation was primarily due to the modified sugar, and not the influence of the base. As a more direct test of this proposition, acyclo-GTP and ddGTP incorporation was compared for 9N™ (exo-)/A485L and Thermo Sequenase™ DNA polymerases (Example 12). These two enzymes gave different responses, with Thermo Sequenase™ showing a preference for the ddGTP substrate, and 9° N™ (exo-)/A485L DNA polymerase showing a preference for acyclo-GTP. Results with the Family A polymerase Thermo Sequenase™ echo those reported in U.S. Pat. No. 5,558,991 for another Family A DNA polymerase, Klenow fragment, in the later case requiring a ten-fold higher acyclo-NTP concentration to see sequencing patterns identical to those obtained with ddNTP triphosphates. Thus, acyclo-NTPs are more efficient terminators than ddNTPs with 9° N™ (exo-)/A485L, and by extension Family B archaeon DNA polymerases.

Three Elements Combine to Enhance Chain Terminator Incorporation

In summary, three elements are identified to enhance chain terminator incorporation. First, use of specific dye adducts attached to the nucleoside base. Second, use of acyclo base analogs. Third, use of archaeon DNA polymerase variants with increased ability to incorporate nucleotides with modified sugars. Each of the three elements alone enhances the desired incorporation. To a first approximation, these elements appear to act in an additive way, with the combined effects of two elements being greater than either alone, and the combined effects of all three elements being greater than any two elements alone.

While dye derivatives are emphasized in this application, the skilled worker will also recognize that other types of modified nucleotides could also be used. For example, the fluorescein moiety can also act as a hapten in antibody-based detection systems. Similarly, other nucleotide modifications that cross-react with a second molecule that can act in a detection scheme will also function in this invention.

While this invention treats incorporation of various terminators into otherwise native DNA on a DNA template, one skilled in the art will recognize that either the template or primer utilized in the reaction may contain nucleotide analogs that allow them to be the functional equivalent of such substrates. Such analogs might include, but not be limited to, thiophosphate backbone linkages, substituted bases and ribonucleotides. The invention requires only that the DNA polymerase employed be able to direct incorporation of the terminator in a base-specific fashion.

One significant advantage arising from more efficient incorporation of dye-terminators is a reduction in the amount of dye-terminator needed in the polymerization reaction. As a consequence, lower backgrounds and increased sensitivity of detection are anticipated due to the higher ratio of incorporated to unincorporated substrate.

Applications of the Invention

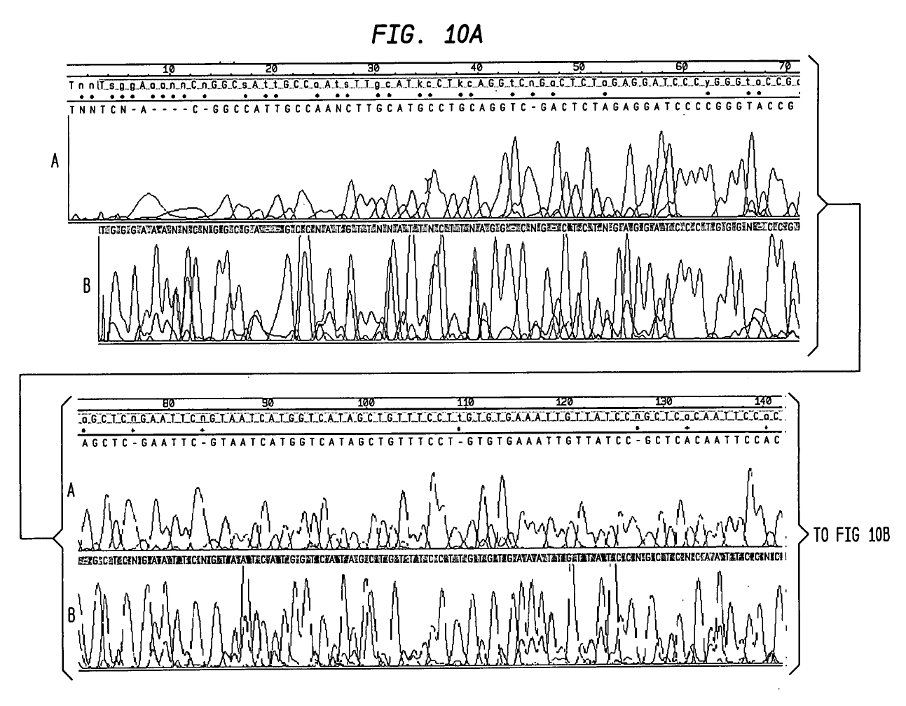

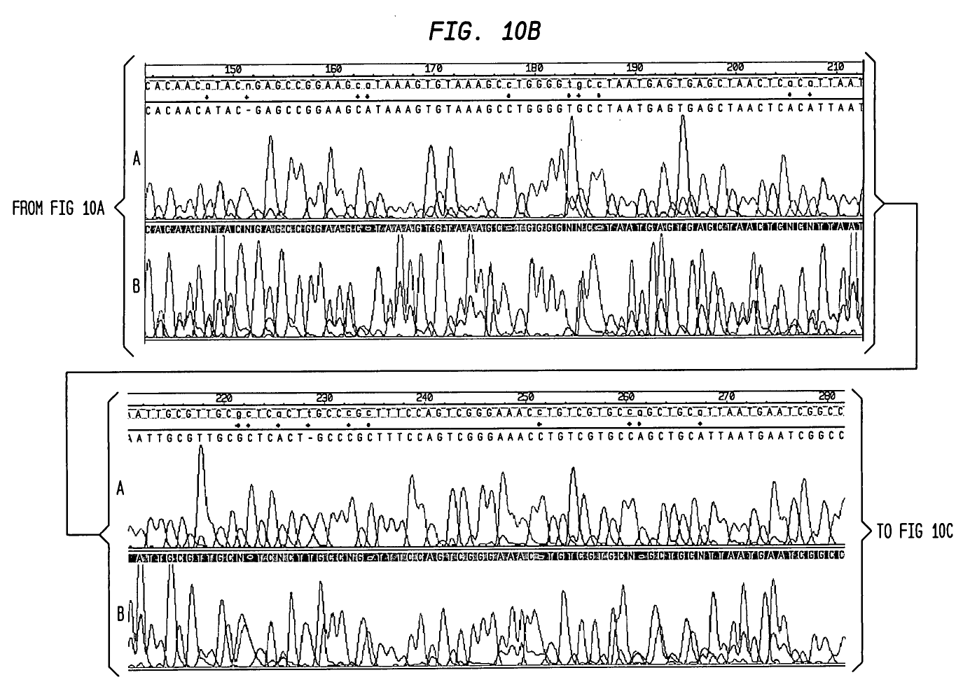

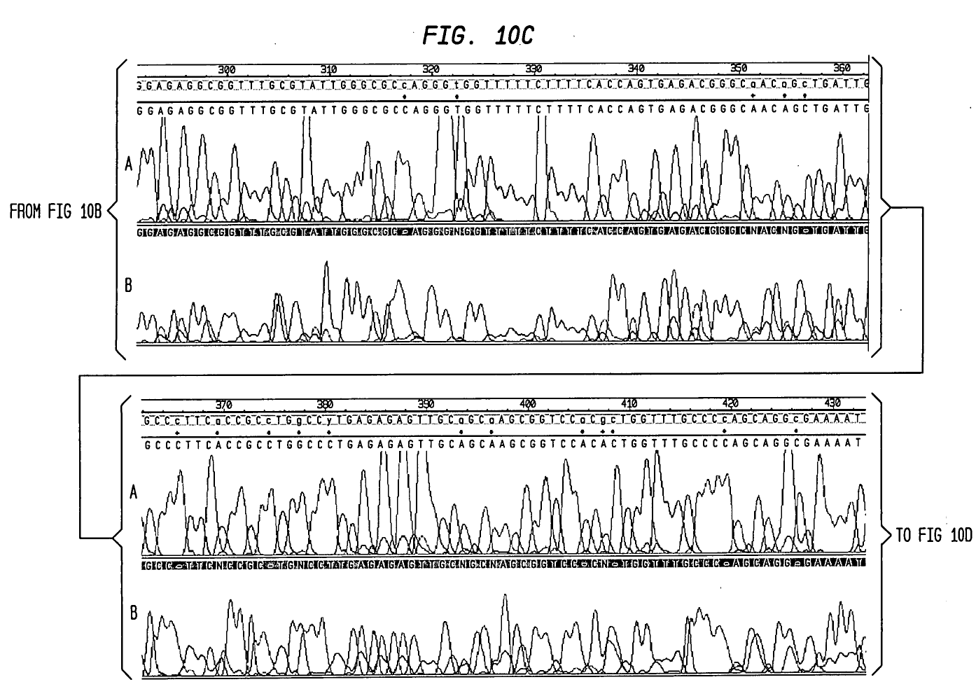

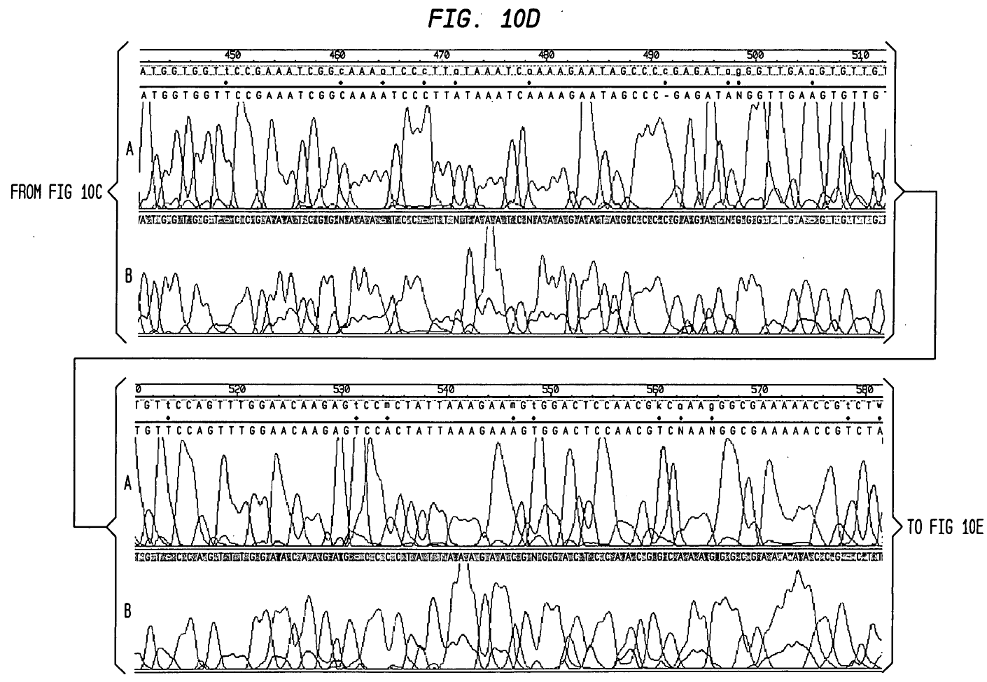

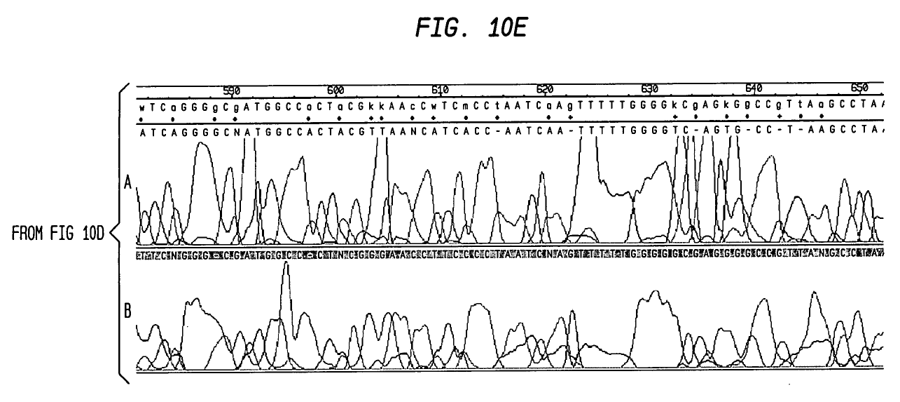

Recognition of the ability of this class of DNA polymerase and polymerase variants to incorporate dye-terminators has a broad range of applications. One obvious application is in DNA sequencing, where incorporation of dye-terminators forms the basis of numerous automated sequencing technologies (Lee, et al., Nucleic Acids Res. 20:2471-2483 (1992); Example 13, FIG. 10). Past experience has shown nucleotide incorporation varies depending on the sequence context, and that this variability is specific for different DNA polymerases (Lee, supra. (1992); Brandis, Nucleic Acids Res. 27:1912-1918 (1999); Parker, Biotechniques 19:116-211 (1995)). It can be reasonably expected that some regions that are difficult to sequence with current protocols could more easily be rendered with the combinations of polymerases and terminators disclosed here. Since the insertion properties of these polymerases are different from those of Taq and related DNA polymerases, it may also be possible to mix archaeon DNA polymerases with other polymerases in sequencing reactions to allow more uniform signal incorporation, with both enzymes contributing to the final sequencing product. This mixing could be extended to other applications where dye-substituted bases are incorporated.

Other related applications such as fluorescence dideoxy finger-printing (F-ddF: Ellison, et al., Biotechniques 17:742-753 (1994)) can make use of this invention. Such applications require only short-range DNA sequence determinations, some as short as a single base. Discovery of compatible combinations of archaeon DNA polymerases and dye terminators allows such determinations to go forward, as might be required in detecting single nucleotide polymorphisms (SNPs; U.S. Pat. Nos. 5,888,819, 5,952,174, 6,004,744, and 6,013,43). For example, allele-specific primers can be extended by dye-labeled terminators, and the specific nucleotide inserted later detected by either fluorescence polarization (Chen, et al., Genome Research 9:492-498 (1999)) or by fluorescence resonance energy transfer (Chen and Kwok, Nucleic Acids Res. 25:347-353 (1997)).

The ability to insert non-standard nucleotides is also useful in sequencing applications employing mass spectroscopy. One limitation of multiplex genotyping by mass spectrometry is distinguishing the masses of oligonucleotide primers extended by a single nucleotide. By increasing the difference in mass between the four nucleotides added, increased resolution could be achieved, allowing analysis of larger oligomers, and increased confidence in multiplex analysis where a large number of different molecular weights will need to be determined (Ross, et al., Nature Biotechnology 16:1347-1351 (1998)). Of course, incorporation of acyclo-derivatives without dyes could also be employed in this application.

The present invention is further illustrated by the following Examples. These Examples are provided to aid in the understanding of the invention and are not construed as limitations thereof.

The references cited above and below are herein incorporated by reference.

### EXAMPLE 1

A Titration Assay to Measure the Relative Efficiency of Modified Nucleotide Incorporation

The relative efficiency of modified nucleotide incorporation was assessed using variations of the assay described by Gardner and Jack (supra.). A primed single-stranded DNA substrate is incubated in a reaction mixture containing a fixed concentration of dNTPs and increasing amounts of the modified nucleotide. Reactions can either be isothermal, or can be linearly amplified by thermal cycling using stages of denaturation, annealing and primer extension. Following the reaction, terminated extension products are separated by denaturing polyacrylamide gel electrophoresis, and the separated products detected either by virtue of labels attached to the primer (e.g., 5′-[³²P] end-labeled) or terminator (e.g., dye-labels) using methods commonly known in the art, such as autoradiography and fluorescent scanning.

Once the spectrum of termination products are determined, a comparison of the length, uniformity and clarity of this pattern is used to evaluate the relative incorporation of the modified nucleotide. The more readily incorporated terminators will produce a given pattern when present in lower concentrations than the comparison standard. Alternatively, the banding pattern at a given concentration of modified nucleotide can be compared between two or several compounds. The compounds producing shorter termination products at a given concentration are those that are more efficiently incorporated by the DNA polymerase. This latter method can theoretically be performed using a single analog concentration, although it is more desirable to use multiple concentrations to provide greater opportunities for comparison.

A control reaction, containing no terminator, confirms that the polymerase is able to fully extend the primer (approximately 7200 bp in the case of M13mp18) in the absence of the terminator. Thus, the bands observed in other reactions arise from terminator incorporation rather than incomplete replication by the DNA polymerase.

### EXAMPLE 2

Dye-ddCTP Derivatives Differ in Incorporation Efficiency

A variety of available dye-labeled ddCTP derivatives (Table 2) were analyzed and compared to test for incorporation by Vent® (exo-) DNA polymerase. Primed M13mp18 substrate was formed as previously described (Kong, et al., supra.). As in all the examples, all reaction components were from New England Biolabs (Beverly, Mass.), except where indicated. Incorporation of modified bases was assayed by mixing 2.5 μl of 2X reaction cocktail (0.04 μM 5′[³²P] end-labeled #1224-primed M13mp18, 2X ThermoPol Buffer, 0.04 U/μp thermostable pyrophosphates, 80 μM dNTP, 0.15 U/μl DNA polymerase) with 2.5 μl of nucleotide analog solution to yield the final ratios of analog:dCTP indicated in the figures. After incubating at 72° C. for 15 minutes, the reactions were halted by the addition of 4 μl CircumVent® stop/dye (0.3% xylene cyanole FF., 0.3% bromophenol blue, 0.37% EDTA, pH 7.0). Samples were then heated at 72° C. for 3 minutes and separated on a QuickPoint DNA sequencing gel (NOVEX, San Diego, Calif.) run at 1200 volts. The gel was fixed by soaking in 10% acetic acid/10% ethanol, dried, and polymerization products visualized by autoradiography. Examples of these reactions are given in FIG. 1.

The extent of dye-terminator was determined by visual inspection of the autogradiograms. Evaluations followed the outlines given in Example 1, and are recorded in Table 2.

TABLE 2

| | | |
|---|---|---|
| Dye-Terminator | Catalog Number | Incorporation |

Commercially available dye-terminators from NEN Life Science
Products Catalog

| | | |
|---|---|---|
| JOE-ddATP | NEL486 | |
| JOE-ddCTP | NEL485 | less |
| JOE-ddGTP | NEL487 | |
| JOE-ddUTP | NEL484 | |
| TAMRA-ddATP | NEL474 | |
| TAMRA-ddCTP | NEL473 | better |
| TAMRA-ddGTP | NEL475 | |
| TAMRA-ddUTP | NEL472 | |
| FAM-ddATP | NEL482 | |
| FAM-ddCTP | NEL481 | less |
| FAM-ddGTP | NEL483 | |
| FAM-ddUTP | NEL480 | |
| ROX-ddATP | NEL478 | |
| ROX-ddCTP | NEL477 | better |
| ROX-ddGTP | NEL486 | |
| ROX-ddUTP | NEL479 | |
| Fluorescein-12-ddATP | NEL402 | |
| Fluorescein-12-ddCTP | NEL400 | |
| Fluorescein-12-ddGTP | NEL403 | |
| Fluorescein-12-ddUTP | NEL401 | |
| R6G-ddCTP | NEL489 | better |
| R110-ddCTP | NEL493 | slightly worse |

Non-commercially available dye-terminators tested

| | |
|---|---|
| ROX-acyclo-CTP | better |
| TAMRA-acyclo-CTP | better |
| FI-12-acyclo-CTP | better |
| IRD40-ddCTP | similar |
| IRD700-ddCTP | slightly better |
| IRD700-acyclo-NTP* | better |
| Cyanine 3-ddCTP | less |
| Cyanine 5-ddCTP | less |
| BODIPY ® TR-ddCTP | better |
| BODIPY ®TMR-ddCTP | better |
| BODIPY ®R6G-ddCTP | similar |
| BODIPY ®FI-ddCTP | similar |
| BODIPY ®FL-acyclo-GTP | better |
| IRD40-acyclo-NTP* | better |
| R6G-acyclo-ATP | better |

Analogs labeled with * were tested for all four bases. Incorporation refers to incorporation relative to ddNTP. These alkynylamino-acyclic analogs are covered by U.S. Pat. Nos. 5,047,519 and 5,151,507 issued to NEN Life Science Products. Inc.

### EXAMPLE 3

Blast Comparison of Family B DNA Polymerases

One method of classifying and categorizing proteins is by primary amino acid sequence alignment. It is generally accepted that high degrees of primary sequence similarity suggest similar function, and thus can be predictive of physical and enzymatic properties common between the compared proteins. A sampling of archaeon DNA polymerases, along with representatives of other Family B and Family A DNA polymerases, were compared using the sequence alignment program Blastp. This program searches for a maximal collinear sequence alignment between test sequences, with output reported in terms of sequence identity, sequence similarity (where paired amino acid residues have similar characteristics), and gaps introduced to maintain the alignment.

The source for sequence information was the ncbi server at the internet site: http://www.ncbi.nlm.nih.gov and accession numbers derived from that site are listed along with the source organism in Table 3. Blastp comparisons were run pairwise with either the Vent® DNA polymerase amino acid sequence,with the following program parameters:

matrix: OBLOSUM62

gap open: 11

gap extension: 1

x_dropoff: 50

expect: 10

wordsize: 3

filter: off

Comparisons were done via the internet site:

http://www.ncbi.nlm.nih.gov/gorf/bl2.html. Column four of Table 3 report % identity/% positives/% gaps for such comparisons. Entries marked “none” returned “no significant similarity found” on blastp analysis.

The polymerases can be assigned to several groups based on this analysis. First, those polymerases with greater than about 70% sequence identity with Vent® DNA polymerase. In the sequences tested, such enzymes were derived from Thermococcus or Pyrococcus species, although examples from other species may also be found. Second, an intermediate group of between about 30 and 70% identity, with examples listed here deriving from Pyrodictium and Methanococcus species. Third, Family B DNA polymerases with less than about 30% identity, including both archaeon, viral and eukaryotic DNA polymerases. Fourth, Family A DNA polymerases with no significant similarity detected by the analysis.

Those DNA polymerases with higher identity percentages are considered to be more likely to share base analog incorporation properties with Vent® DNA polymerase. The archaeon DNA polymerases reported in later examples derive from the first group, consistent with this prediction. The next most likely group to contain similar sequence characteristics are those with about 30-70% sequence identity. Similarly, the Family A DNA polymerases are shown to have very different properties, as might be suggested by the lack of sequence identity detected by this sequence analysis. The intermediate groups, including the second and third groups above, would be expected to behave more like Vent® DNA polymerase than like the Family A DNA polymerases.

Table 3 also lists an outgrowth of the sequence comparisons, namely an alignment of the conserved region mutagenized in several of the polymerases (see also Table 1). Notable is the conservation of the key, underlined residues in polymerases with high sequence identity to Vent® DNA polymerase, encouraging the view that homologous mutations in those related polymerases will have similar effects on nucleotide incorporation as observed in Vent® DNA polymerase.

### EXAMPLE 4

Purification of 9° N™ (exo-) DNA polymerase

The 9° N™ (exo-) DNA polymerase (referred to as the “AIA” mutant) was constructed, grown and expressed in a T7 expression system as described in Southworth, et al. (supra.). Purification followed the general outline described in that reference and, except where noted, was at 0-4° C. The cell pellet (380 g) was suspended in 1.14 liter of Buffer A (20 mM KPO₄ (pH 6.8), 0.1 mM EDTA, 0.05% Triton X-100, 0.1 M NaCl, 10% glycerol). Cells were lysed by multiple passages through a Manton Gaulin homogenizer, using a cooling coil to keep the homogenate temperature below 20° C. The extract was clarified by centrifugation for 40 min. in a Sharples Type 16 centrifuge at 15,000 rpm (Fraction I, volume 1.5 liters).

The cleared extract was heated to 75° C. for 10 minutes, and then cooled on ice. Insoluble material was removed by centrifugation for 35 min. at 4 krpm in a Beckman JS-4.2 rotor (Fraction II, 0.85 liters).

Fraction II was passed through a 0.7 liter (9.5×10 cm) DEAE-cellulose column, equilibrated in Buffer A containing 1 mM DTT and immediately applied to a 235 ml (5×12 cm) phosphocellulose column equilibrated in the same buffer. The latter column was washed with 0.5 liter of buffer A containing 1 mM DTT, and eluted with a 2 liter linear gradient of NaCl (0.1 -1.0 M). Polymerase activity was assayed, and peak fractions pooled (Fraction III, volume 0.4 liter, approximately 0.8 g protein).

Fraction III was dialyzed against buffer B (20 mM TrisHCI (pH 7.6), 0.1 M NaCl, 1 mM DTT, 0.1 mM EDTA, 10% glycerol), and passed through a 49 ml (2.5×10 cm) DEAE-cellulose column. The column was washed with 50 ml of buffer B, and the wash was combined with the flow-through fractions (Fraction IV, volume 0.45 liter).

Fraction IV was dialyzed against buffer C (20 mM TrisHCl (pH 7.5), 0.05 M NaCI, 1 mM DTT, 0.1 mM EDTA, 10% glycerol). Solid (NH₄)₂SO₄ was added (101 g) to a final concentration of 1.7 M, and the solution was filtered through a 0.22 μm filter. The filtered solution was applied to a 0.05 liter phenyl-sepharose column (2.5×10 cm) equilibrated in buffer C containing 1.7 M (NH₄)₂SO_{4.} The column was washed with 0.1 liter of buffer C containing 1.7 M (NH₄)₂SO_{4,} and eluted with a 0.5 liter linear gradient of (NH₄)₂SO₄ (1.7-0 M) in buffer C. Column fractions were assayed for polymerase activity and fractions containing the peak of polymerase activity were pooled (Fraction V, 90 ml, approximately 0.3 g protein).

Fraction V was dialyzed against buffer B, and loaded onto a 53 ml (2.5×10 cm) Affigel Blue column equilibrated in the same buffer. Following loading, the column was washed with 0.1 liter of buffer B, and eluted with a 0.5 liter linear gradient of NaCI (0.1-1.35 M) in buffer B. Fractions were assayed for polymerase activity, and the peak of the activity fractions were pooled and dialyzed into storage buffer (10 mM TrisHCl (pH 7.4), 0.1 M KCl, 0.1 mM EDTA, 1 mM DTT, 0.1% Triton X-100, 50% glycerol) and stored at −20° C. (Fraction VI, 40 ml, approximately 0.15 g protein, 1.2×10⁶ units).

### EXAMPLE 5

Dye-acyclo-CTP Derivatives are more efficiently Incorporated than dye-ddCTP Derivatives by Vent® (exo-) DNA Polymerase

Acyclo-NTPs, similar to ddNTPs, lack a free 3-OH termini, and are expected to act as chain terminators in DNA polymerase reactions. The ability of acyclo-NTPs with dye-derivatized bases to act as chain terminators was tested using a titration assay. Incorporation of ROX-ddCTP, TAMRA-ddCTP and IRD700-ddCTP were compared to that of ROX-acyclo-CTP, TAMRA-acyclo-CTP and IRD700-acyclo-CTP, respectively, using Thermo Sequenase™ and Vent® (exo-) DNA polymerases.

Incorporation of modified bases was assayed by mixing 2.5 μl of 2X reaction cocktail (0.04 pM 5′ [³²P] end-labeled #1224-primed M13mpl18, 2X Thermopol Buffer, 0.04 U/μp thermostable pyrophosphates, 80 μM dNTP, 0.15 U/μl DNA polymerase) with 2.5 μl of nucleotide analog solution to yield the final ratios of analog:dCTP indicated in the figures. After incubating at 72° C. for 15 minutes, the reactions were halted by the addition of 4 μl CircumVent® stop/dye (0.3% xylene cyanole FF., 0.3% bromophenol blue, 0.37% EDTA (pH 7.0)). Samples were then heated at 72° C. for 3 minutes and separated on a QuickPoint DNA sequencing gel (NOVEX, San Diego, Calif.) run at 1200 volts. The gel was fixed by soaking in 10% acetic acid/10% ethanol, dried, and polymerization products visualized by autoradiography. Examples of these reactions are given in FIG. 2.

A comparison of banding patterns revealed shorter terminator products for the acyclo-CTP, as opposed to the ddCTP, derivatives when using Vent® (exo-) DNA polymerase. This was the case for ROX, TAMRA and IRD700 derivatives. Thus, the acyclo derivatives are more efficiently incorporated than their dideoxy equivalents. In contrast, Thermo Sequenase™ showed a preference for dideoxy derivatives, as evident in the collection of shorter termination products for those ROX, TAMRA and IRD700 derivatives.

### EXAMPLE 6

Sensitivity to dye-acyclo-CTP Terminators is Shared by a Variety of Archaeon DNA Polymerases

The sequence similarity among archaeon Family B DNA polymerases raises the possibility that they will function similar to Vent® (exo-) DNA polymerase with respects to incorporation of dye terminators. This proposition was tested using ROX-acyclo-CTP in a titration assay to compare the performance of Vent® (exo-), Deep Vent® (exo-), Pfu (exo-) and 9° N™ (exo-) DNA polymerases. A titration assay of the type described in Example 1 was used.

Briefly, a 2X reaction cocktail was prepared on ice containing 0.06 μM 5-[³²P] #1224 primed single-stranded M13mp18, 2X ThermoPol Buffer (20 mM KCl, 40 mM Tris-HCl (pH 8.8 at 25° C.), 20 mM (NH₄)₂SO₄, 4 mM MgSO₄, 0.2% Triton X-100), 0.04 U/μl thermostable inorganic pyrophosphatase and 80 μM dNTP. The 2X cocktail was split into aliquots and Vent® (exo-), Deep Vent® (exo-) or 9° N™ (exo-) DNA polymerase was added to a final concentration of 0.06 U/μl. Another 2X cocktail was made with conditions recommended by the manufacturer (Stratagene, La Jolla, Calif.) for Pfu (exo-) DNA polymerase containing 0.06 μM 5 ′-[³²P] #1224 primed single-stranded M13mp18, 2X Pfu Buffer (20 mM KCl, 20 mM (NH₄)₂SO₄, 40 mM Tris-HCl (pH 8.75), 4 mM MgSO₄, 0.2% Triton X-100, 0.2 mg/ml BSA), 0.04 U/μl thermostable inorganic pyrophosphatase, and 80 μM dNTP to which Pfu (exo-) DNA polymerase was added to a final concentration of 0.06 U/μl. A 2.5 μl aliquot of 2X reaction cocktail was mixed with 2.5 μl of nucleotide analog to yield the final ratios of analog:dCTP indicated in the figures. Control extensions added 2.5 μl of dH₂O to 2.5 μl of reaction mix, and demonstrated that polymerization proceeded without termination in the absence of ROX-acyclo-CTP. Following mixing, reactions were immediately incubated at 72° C. for 20 minutes.

Reactions were stopped by the addition of 4 μl NEB Stop/Loading Dye Solution (deionized formamide containing: 0.3% xylene cyanole FF, 0.3% bromophenol blue, 0.37% EDTA (pH 7.0)) and heated at 72° C. for 3 minutes. A 1 μl aliquot was loaded onto a QuickPoint (NOVEX) mini-sequencing gel and run at 1200 V for 10 minutes. The gel was fixed, washed, and dried according to manufacturer's instructions and polymerization products visualized by autoradiography (FIG. 3).

Based on analysis of termination fragment lengths, ROX-acyclo-CTP was incorporated by all four archaeon DNA polymerases Vent®) (exo-), Deep Vent® (exo-), Pfu (exo-) and 9° N™ (exo-). At high concentrations (2: 1 molar ratio) of ROX-acyclo-CTP:dCTP, termination products were very short due to the efficient incorporation of the terminator. The similar range and quality of termination fragments suggesting that all four archaeon DNA polymerases incorporate ROX-acyclo-CTP with comparable efficiency.

### EXAMPLE 7

Incorporation of dye-labeled Terminators is Enhanced by Vent® (exo-)/A488L DNA polymerase

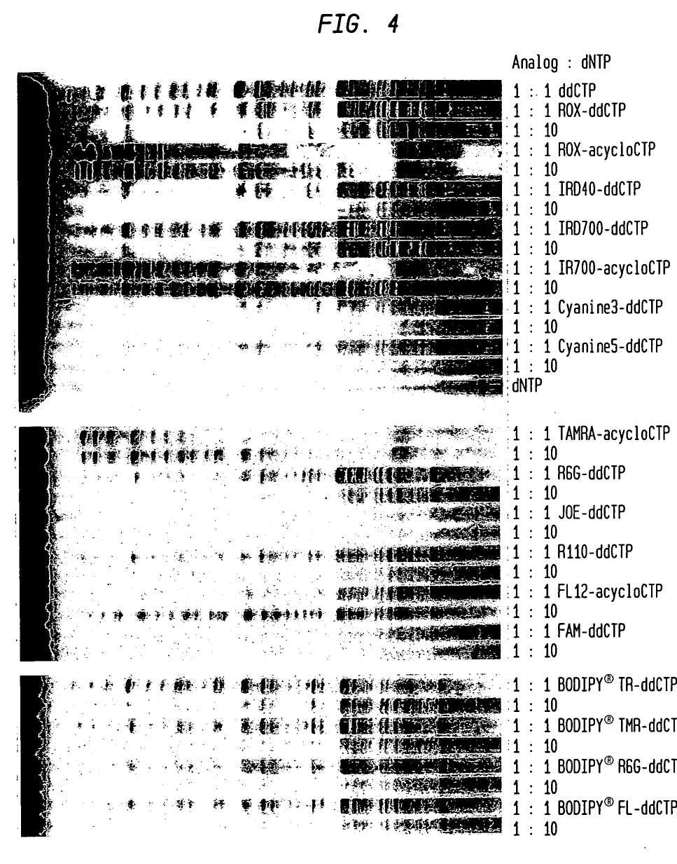

The ability of Vent® (exo-)/A488L DNA polymerase to incorporate dye-labeled terminators was evaluated using the titration assay described in Example 1. A variety of available dye-labeled ddCTP derivatives (Table 2) were analyzed and compared to test for incorporation by Vent® (exo-) DNA polymerase. Primed M13mp18 substrate was formed as previously described (Kong, et al., supra.). As in all the examples, all reaction components were from New England Biolabs (Beverly, Mass.), except where indicated. Incorporation of modified bases was assayed by mixing 2.5 μl of 2X reaction cocktail (0.04 μM 5′ [^{32 P] end-labeled #}1224-primed M13mp18, 2X Thermopol Buffer, 0.04 U/μl thermostable inorganic pyrophosphatase, 80 μM dNTP, 0.15 U/μl DNA polymerase) with 2.5 μl of nucleotide analog solution to yield the final ratios of analog:dCTP indicated in the figures. After incubating at 72° C. for 15 minutes, the reactions were halted by the addition of 4 μl CircumVent® stop/dye (0.3% xylene cyanole FF., 0.3% bromophenol blue, 0.37% EDTA, pH 7.0). Samples were then heated at 72° C. for 3 minutes and separated on a QuickPoint DNA sequencing gel (NOVEX, San Diego, Calif.) run at 1200 volts. The gel was fixed by soaking in 10% acetic acid/10% ethanol, dried, and polymerization products visualized by autoradiography. Examples of these reactions are given in FIG. 4.

In each case dye-terminators were incorporated more efficiently by Vent® (exo-)/A488L DNA polymerase than by the parent Vent® (exo-) DNA polymerase. Despite this increase, the relative efficiency of incorporation among the various dye-terminators was the same for both enzymes.

### EXAMPLE 8

Alternate DNA polymerase variants can also enhance the incorporation of terminators

Additional Vent® (exo-) DNA polymerase variants were also tested for their ability to enhance incorporation of dye-terminators. The variant Y499L was compared with the parental (exo-) polymerase, along with the A488L variant in its ability to incorporate ROX-ddCTP.

As in previous examples, the efficiency of analog incorporation was determined using a titration assay, using varying concentrations of terminators. Briefly, a 2X reaction cocktail was prepared on ice containing 0.04 μM single-standed M13mp18 primed with 5 ′[³²P] end-labeled #1224 primer, 2X ThermoPol Buffer (20 mM KCl, 40 mM Tris-HCl (pH 8.8 at 25° C.), 20 mM (NH₄)₂S0₄, 4 mM MgSO₄, 0.2% Triton X-1 00), 0.04 U/pl thermostable inorganic pyrophosphatase and 80 μM dNTP. The 2X cocktail was split into aliquots and Vent® (exo-), Vent® (exo-)/A488L or Vent® (exo-)/Y499L DNA polymerase was added to a final concentration of 0.06 U/μl. A 2.5 μl aliquot of this 2X reaction cocktail was mixed with 2.5 μl of a nucleotide analog mixture, resulting in the final ratios of analog:dCTP indicated in the figures. Control reactions mixed 2X reaction cocktail with an equal volume of dH₂O. Reactions were immediately incubated at 72° C. for 15 minutes.

Reactions were stopped by the addition of 4 μl NEB Stop/Loading Dye Solution (deionized formamide containing: 0.3% xylene cyanole FF, 0.3% bromophenol blue, 0.37% EDTA (pH 7.0)) and heated at 72° C. for 3 minutes. A 1 μl aliquot was loaded onto a QuickPoint (NOVEX) mini-sequencing gel and run at 1200 V for 10 minutes. The gel was then fixed, washed, and dried according to manufacturer's instructions and reaction products were visualized by autoradiography (FIG. 5).

Both the A488L and Y499L variants of Vent® (exo-) DNA polymerase were better able to incorporate ROX-ddCTP than the parent Vent® (exo-) DNA polymerase (FIG. 5) as evidenced in the shorter termination products produced by those variants at the same analog concentrations. Incorporation by the two variants was comparable, with slightly more efficient terminator incorporation by the A488L variant. Approximately 5-10-fold lower concentrations of ROX-ddCTP were required to produce equivalent banding patterns with the variants compared to the parent enzyme. These enzymes are thus useful tools for greater incorporation of chain terminating nucleotides.

### EXAMPLE 9

Generation of 9° DNA Polymerase Variants

Production and purification of Vent® DNA polymerase variants was as described (Gardner and Jack, supra). This led to enzyme preparations that were substantially purified, meaning separated from contaminants affecting the performance of the enzyme, such as contaminating exo- and endonucleases, alternate polymerases and endogenous nucleotides. Purification of 9° N™ (exo-) DNA polymerase and the A485L variant of that enzyme used the same protocols.

An expression vector for the A485L variant of 9° N™ DNA polymerase was created using PCR mutagenesis (Colosimo, et al. Biotechniques 26:870-873 (1999)) of the expression construct pNEB917, a derivative of pNEB915 encoding an exonuclease-deficient (AIA) form of the polymerase (Southworth, et. al, Proc. Natl. Acad. Sci. USA 93:5281-5285 (1996)).

The mutagenesis used two successive PCR reactions. The first stage reactions (0.05 ml) contained 1 X Thermopol buffer (New England Biolabs, Beverly, Mass.), 50 ng/ml pNEB915 template DNA, 0.25 mM dNTPs, 0.5 μM oligonucleotide #216-153 (SEQ ID NO:1; Table 4), 0.5 μM oligonucleotide #175-70 (SEQ ID NO:2; Table 4), 0.1 mg/ml bovine serum albumen and 2 mM added MgSO₄ in a 0.2 ml thin-wall PCR tubes. One unit of Vent® DNA polymerase was added to the reaction mixture, and the tube containing the mixture was heated at 94° C. for three minutes, followed by 25 cycles of 94° C. (15 seconds), 58° C. (15 seconds), 72° C. (60 seconds). Evaluation of the reaction product on an agarose gel revealed a band of the expected molecular weight.

TABLE 4
Oligonucleotide Sequences

| | |
|---|---|
| Oligo # | Sequence |

| | |
|---|---|
| 216-153 | CAGGCAGAGGCTTATAAAAATCCTCGCCAACAGCTT |
| | (SEQ ID NO:1) |
| 175-70 | GGTGGCAGCAGCCAACTCAGCTTCCT (SEQ ID NO:2) |
| 216-155 | GATTCTCATGATAAGCTACGCCGA (SEQ ID NO:3) |

The second round of PCR was accomplished by diluting the above PCR sample either 250- or 500-fold into 0.1 ml reaction mixtures containing: 1X Thermopol buffer, 0.1 mg/ml bovine serum albumen, 0.25 mM dNTPs, 0.5 μM oligonucleotide #175-70 (SEQ ID NO:2; Table 4), 0.5 μM oligonucleotide #216-155 (SEQ ID NO:3; Table 4), and 0, 2, 4, 6 or 8 mM added MgSO₄, again in 0.2 ml thin-walled PCR tubes. After addition of one unit of Vent® DNA polymerase to each reaction mixture, the sample was heated at 94° C. for three minutes, followed by 25 cycles of 94° C. (15 seconds), 58° C. (15 seconds), 72° C. (90 seconds). Aliquots of each reaction were analyzed by agarose gel electrophoresis and found to contain a band of the expected size (about 1.5 kb). Samples containing 2-8 mM added MgSO₄ were pooled, phenol extracted and ethanol precipitated.

The precipitated sample was suspended in 0.1 ml of 1 X NEBuffer 2, and cut sequentially with the restriction endonucleases BamHl (100 units for 1 hour at 37° C.) and BsiWl (75 units for 1 hour at 55° C.). The plasmid pNEB917 was similarly digested with the same enzymes. The reaction products from both samples were separated on a 0.7% agarose gel in TBE buffer containing 0.5 pg/ml ethidium bromide. The prominent approximately 1.5 kb band derived from the PCR sample and the approximately 7 kb band derived from pNEB917 were excised and eluted using an Elutrap apparatus in 0.5X TBE, using conditions specified by the manufacturer (Schliecher & Schuell, Keene, N.H.). The eluted DNAs were phenol extracted and ethanol precipitated. After suspension of the DNA pellet in TE buffer, the samples were quantified by running small aliquots on an agarose gel, and comparing the samples with molecular mass and weight standards.

The eluted fragments were ligated, and ampicillin resistant transformants were selected and screened by cleavage with Psil, a site not present on pNEB917, but which would be gained if the mutagenesis was successful. One construct displaying the Psil site was named pEAC3, and was used for expression of 9° N™ (exo-/A485L) DNA polymerase.

Expression and purification of the variant DNA polymerase was as described for Vent® (exo-)/A488L (Gardner and Jack, supra.).

### EXAMPLE 10

Vent® (exo-)/A488L and 9° N™ (exo-)/A485L DNA polymerases both efficiently incorporate ROX-ddCTP

The high degree of sequence identity in archaeon DNA polymerases suggests that variants similar to the A488L variant described in Example 7 should function similarly with respects to dye-terminator incorporation. Accordingly, Vent® (exo-)/A488L and 9° N™ (exo-)/A485L DNA polymerases were compared in their ability to incorporate both ddCTP and ROX-ddCTP.

A 2X reaction cocktail was prepared on ice containing 0.04 μM single-stranded M13mp18 primed with 5 ′-[³²P] end-labeled #1224 primer, 2X ThermoPol Buffer (20 mM KCl, 40 mM Tris-HCl (pH 8.8 at 25° C.), 20 mM (NH₄)₂SO₄, 4 mM MgSO₄, 0.2% Triton X-100), 0.04 U/μl thermostable inorganic pyrophosphatase and 80 μM dNTP. The 2X cocktail was split into aliquots and Vent® (exo-), Vent® (exo-)/A488L or 9° N™ (exo-)/A485L DNA polymerase was added to a final concentration of 0.04 U/μl. A 2.5 μl aliquot of this 2X reaction cocktail was mixed with 2.5 μl of a nucleotide analog mixture, resulting in the final ratios of analog:dCTP indicated in the figures. Control reactions mixed 2X reaction cocktail with an equal volume of dH₂O. Reactions were immediately incubated at 72° C. for 15 minutes.

Reactions were stopped by the addition of 4 μl NEB Stop/Loading Dye Solution (deionized formamide containing: 0.3% xylene cyanole FF, 0.3% bromophenol blue, 0.37% EDTA (pH 7.0)) and heated at 72° C. for 3 minutes. A 1 μl aliquot was loaded onto a QuickPoint (NOVEX) mini-sequencing gel and run at 1200 V for 10 minutes. The gel was then fixed, washed, and dried according to manufacturer's instructions and reaction products were visualized by autoradiography (FIG. 6).

Reactions of Vent® DNA polymerase (exo-) and 9° N™ (exo-) with ddCTP were characterized by a faint series of high molecular weight bands, reflecting relatively weak incorporation of ddCTP under these conditions. The same pattern of termination products is observed when ROX-ddCTP is used at approximately 25-fold lower concentrations, a measure of the increased efficiency of incorporation of the ROX analog by this enzyme.

The 9N™ (exo-)/A485L DNA polymerase variant mimicked the enhanced incorporation noted for the Vent® (exo-)/A488L DNA polymerase both with respects to relative incorporation of ddCTP and ROX-ddCTP (FIG. 6). With each enzyme depicted in FIG. 6, ROX-ddCTP is incorporated about 10-fold better than ddCTP (compare 10:1 vs.1:1 lanes for ddCTP:dCTP vs. ROX-ddCTP:dCTP, respectively). Additional comparison of the parental and variant enzymes showed a further approximately 10-fold enhancement of either ROX-ddCTP or ddCTP incorporation.

### EXAMPLE 11

Archaeon DNA Polymerase Variants Display Enhanced Incorporation of dye-acyclo-NTPs Relative to dye-ddNTPs

While the previous examples illustrate the utility of using archaeon DNA polymerase variants to enhance incorporation of dye-ddNTP terminators, their utility in incorporation of dye-acyclo-NTPs was unknown. The experiments in this example illustrate that terminator incorporation is further enhanced when archaeon variants are used in combination with acyclo terminators. Two sets of experiments established these facts.

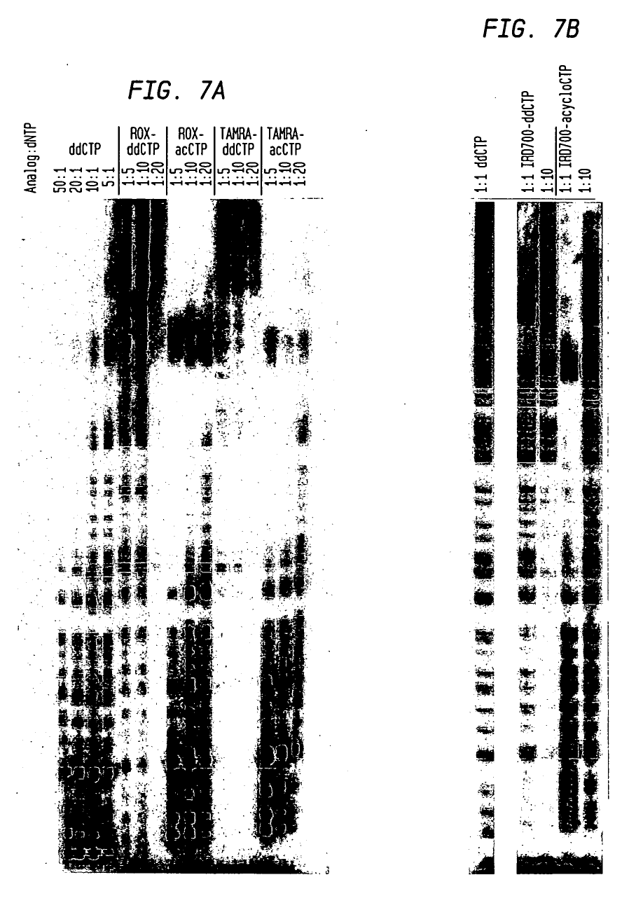

In the first experiment, a 2X reaction cocktail was prepared on ice containing 0.04 pM single-stranded M13mp18 primed with 5′-[³²P] end-labeled #1224 primer, 2X ThermoPol Buffer (20 mM KCl, 40 mM Tris-HCl (pH 8.8 at 25° C.), 20 mM (NH₄)₂SO₄, 4 mM MgSO₄, 0.2% Triton X-100), 0.04 U/μl thermostable inorganic pyrophosphatase and 0.08 mM dNTP. The 2X cocktail was split into aliquots and Vent® (exo-)/A488L DNA polymerase was added to a final concentration of 0.04 U/μl (0.08 U/μl for IRD700-acyclo-CTP). A 2.5 μl aliquot of this 2X reaction cocktail was mixed with 2.5 μl of a nucleotide analog mixture, resulting in the final ratios of analog:dCTP indicated in FIG. 7. Reactions were immediately incubated at 72° C. for 20 minutes.

Reactions were stopped by the addition of 4 μl NEB Stop/Loading Dye Solution (deionized formamide containing: 0.3% xylene cyanole FF, 0.3% bromophenol blue, 0.37% EDTA (pH 7.0)) and heated at 72° C. for 3 minutes. A 1 μl aliquot was loaded onto a QuickPoint (NOVEX) mini-sequencing gel and run at 1200 V for 10 minutes. The gel was then fixed, washed, and dried according to manufacturer's instructions and reaction products were visualized by autoradiography (FIG. 7).

As noted with the parental Vent® (exo-) DNA polymerase, incorporation of dye-acyclo-CTP was enhanced relative to dye-ddCTP for IRD700, ROX and TAMRA analogs. Thus, this variant retains the favorable acyclo-NTP incorporation characteristics of the parent.

In a related experiment dye-acyclo-CTP incorporation by the analogous 9°0 N™(exo-)/A485L variant was evaluated. A 2X reaction cocktail was prepared on ice containing 0.1 mg/ml single-stranded M13mp18, 0.1 μM 5′[³²P] labeled primer #1224, 2X ThermoPol Buffer (20 mM KCl, 40 mM Tris-HCl (pH 8.8 at 25° C.), 20 mM (NH₄)₂SO₄, 4 mM MgSO₄, 0.2% Triton X-100), 0.04 U/μl thermostable inorganic pyrophosphatase and 80 μM dNTP. The 2X cocktail was split in half and Vent® (exo-)/A488L, 9°0 N™ (exo-)/A485L or Thermo Sequenase™ DNA polymerase was added to a final concentration of 0.06 U/μl. A 2.5 μl aliquot of 2X reaction cocktail was mixed with 2.5 μl of an 80μM nucleotide analog mix in 0.5 ml tubes and immediately incubated at 72° C. for 20 minutes.

Reactions were stopped by the addition of 4 μl NEB Stop/Loading Dye Solution (deionized formamide containing: 0.3% xylene cyanole FF, 0.3% bromophenol blue, 0.37% EDTA (pH 7.0)) and heated at 72° C. for 3 minutes. A 1 μl aliquot was loaded onto a QuickPoint (NOVEX) mini-sequencing gel and run at 1200 V for 10 minutes. The gel was then fixed, washed, and dried according to manufacturer's instructions and reaction products were visualized by autoradiography (FIG. 8).

Vent® and 9° N™ DNA polymerase variants displayed comparable incorporation of all dye-acyclo-CTP analogs tested, establishing the interchangeability of these analogous variants for this invention.

### EXAMPLE 12

Acyclo-GTP is more Efficiently Incorporated than ddGTP by Archaeon DNA Polymerases

Example 5 illustrated the increased efficiency of dye-acyclo-NTPs compared to the corresponding dye-ddNTPs. While these results strongly suggest that the increase in incorporation efficiency arises from the acyclo modification, a direct test was performed. The ability of both Thermo Sequenase™ and 9° N™ (exo-)/A485L to incorporate acyclo-GTP and ddGTP was evaluated using the titration assay.

A 2X reaction cocktail was prepared on ice containing 0.1 mg/ml single-stranded M13mp18, 0.1 μM 5′-³²P] end-labeled primer #1224, 2X ThermoPol Buffer (20 mM KCl, 40 mM Tris-HCl (pH 8.8 at 25° C.), 20 mM (NH₄)₂SO₄, 4 mM MgSO₄, 0.2% Triton X-100), 0.04 U/μl thermostable inorganic pyrophosphatase and 0.1 mM dNTPs. The 2X cocktail was split in half and 9° N™ (exo-)/A485L DNA polymerase or Thermo Sequenase™ was added to a final concentration of 0.04 U/μl. A 2.5 μl aliquot of 2X reaction cocktail was mixed with 2.5 μl of a nucleotide analog mix to yield the final ratios of analog:dGTP indicated in the figures, and immediately placed in a thermal cycler preheated to 94° C. Reactions were thermal cycled as follows:
94° C. 5 minutes
25 cycles at:
   94° C. 30 seconds
   55° C. 30 seconds
   72° C. 30 seconds
   72° C. 7 minutes

Reactions were stopped by the addition of 4 μl Stop/Loading Dye Solution (deionized formamide containing: 0.3% xylene cyanole FF, 0.3% bromophenol blue, 0.37% EDTA (pH 7.0)) and heated at 72° C. for 3 minutes. A 1 μl aliquot was loaded onto a QuickPoint (Novex) mini-sequencing gel and run at 1200 V for 10 minutes. The gel was then fixed, washed, and dried according to manufacturer's instructions and analyzed by autoradiography.

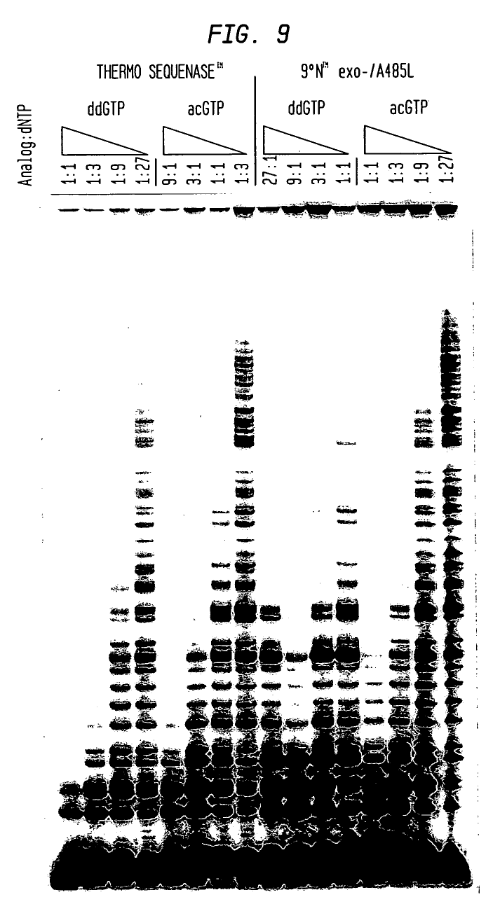

The relative incorporation efficiency of ddGTP and acyclo-GTP by these two DNA polymerases was indicated by the analog concentration yielding equivalent banding pattems (FIG. 9). For example, in a reaction using Thermo Sequenase™, 3:1 acyclo-GTP gave similar banding pattern to that seen with 1:9 ddGTP, indicating an approximate 27-fold preference for ddGTP over acyclo-GTP in these assays. On the other hand, 9° N™ (exo-) /A485L displayed similar banding patterns with 3:1 ddGTP and 1:3 μM acyclo-GTP, indicating an approximately 9-fold preference for acyclo-GTP over ddGTP in these assays.

### EXAMPLE 13

DNA Sequence Analysis Using Archaeon DNA Polymerase Variants and dye-labeled acyclo-NTPs

The enhanced incorporation of modified nucleotides noted in the examples enables a new system of polymerases and reagents for use in automated DNA sequencing. These reactions rely upon incorporation of four chain terminators, each corresponding to one of the four bases normally present in DNA, and each labeled with a uniquely detectable fluorescent dye. The feasibility of such a reaction was tested using the following dye-labeled acyclo-NTPs:R6G-acATP (green), ROX-acCTP (red), BODIPY FL-acGTP (blue), and TAM-acUTP (yellow), where the color indicates the spectrum of the fluorescent emission and “ac” indicates acyclo derivatives. All analogs were obtained from NEN Life Science. Reaction products were separated by denaturing polyacrylamide gel electrophoresis, and detected via fluorescence upon laser excitation.

A reaction cocktail was prepared consisting of 50 ng/μl single-stranded M13mp18, 1 μM #1224 primer, 50 mM TrisHCl (pH 8.0 at room temperature), 8 mM MgSO₄, 0.2 M KCl, 0.1 mM dNTP, 0.1 μM R6G-acATP, 0.1 μM ROX-acCTP, 0.1 μM BODIPY® FL-acGTP, 0.25 μM TAM-acU 0.02 U/μl thermostable inorganic pyrophosphatase and 0.04 U/μl 9° N™ (exo-)/A485L. Reactions were thermal cycled:

94° C. 5 minutes

20 cycles of:
94° C. 30 seconds
58° C. 30 seconds
72° C. 30 seconds
72° C. 7 minutes

AmpliTaq® DNA polymerase, FS reactions were performed using materials acquired from and reaction conditions specified by the manufacturer (ABl PRISM™ Dye Terminator Cycle Sequencing Ready Reaction Kit protocol manual, P/N 402078 Revision A, August 1995, Perkin Elmer Corporation).

After the listed thermal cycling, unincorporated dye-labeled nucleotide terminators were separated by gel filtration using mini-columns (CentriSep, Princeton Separations), lyophilized and suspended in 5 μl formamide stop/dye (deionized formamide containing:0.3% xylene cyanole FF, 0.3% bromophenol blue, 0.37% EDTA (pH 7.0)). Reactions were loaded onto a 4.75% urea gel, and reaction products were separated and detected by an ABl377 automated DNA sequencer. DNA sequencing traces were processed and displayed using software Factura 2.0.1 and AutoAssembler 1.4.0 (Perkin-Elmer Corp.).

Termination fragments are detected by laser-excited fluorescent emission and plotted according to mobility, resulting in a pattern of peaks corresponding to each of the four dye terminators. The color of the peaks corresponds to the dye-acycloNTP that terminates the product. For example, a red peak on the trace would correspond to a product terminated by ROX-acCTP. Software assignment of peak identity appears above traces for both AmpliTaq® DNA Polymerase, FS and 9° N™ (exo-)/A485L reactions, with the anticipated sequence appearing on the top line.

What is claimed is:

1. A method for site-specific incorporation of derivatized dideoxynucleotides into DNA comprising reacting an archaeon Family B DNA polymerase, a primed DNA template and nucleotide solution containing at least one derivatized dideoxynucleotide to produce fragments of DNA with the derivatized dideoxynucleoside covalently attached to the 3′ terminal residue wherein the derivatized dideoxynucleotide is incorporated more efficiently than the corresponding underivatized dideoxynucleotide.

2. A method for site-specific incorporation of acyclonucleotides into DNA comprising reacting an archaeon Family B DNA polymerase, a primed DNA template and nucleotide solution containing at least one acyclonucleotide to produce fragments of DNA with the acyclonucleotide covalently attached to the 3′ terminal residue.

3. A method for site-specific incorporation of derivatized acyclonucleotides into DNA comprising reacting an archaeon Family B DNA polymerase, a primed DNA template and nucleotide solution containing at least one derivatized acyclonucleotide to produce fragments of DNA with the derivatized acyclonucleotide covalently attached to the 3′ terminal residue.

4. The method of claims 1 or 3 where the derivative comprises a detection reagent.

5. The method of claims 1 or 3 where the derivative comprises a dye-label.

6. The method of claims 1 or 3 where the derivative comprises a dye selected from the group consisting of TAMRA, ROX, R6G, Fluorescein-12, IRD40, IRD700, BODIPPY®TR, BODIPY®TMR, BODIPY®R6G and BODIPY®FI.

7. The method of claims 2 or 3 where the acyclonucleotide is radioactively labeled.

8. The method of claims 1-3 wherein the DNA polymerase has at least about 20% primary amino acid sequence identity with Vent® DNA polymerase.

9. The method of claims 1-3 wherein the DNA polymerase has at least about 30% primary amino acid sequence identity with Vent® DNA polymerase.

10. The method of claims 1-3 wherein the DNA polymerase has at least about 70% primary amino acid sequence identity with Vent® DNA polymerase.

11. The method of claims 1-3 wherein the DNA polymerase binds to an antibody probe that has antigenic specificity to Vent® DNA polymerase.

12. The method of claims 1-3 wherein the DNA polymerase is encoded by an isolated DNA fragment that hybridizes in a Southern blot to an isolated DNA fragment selected from the group consisting of a DNA fragment having nucleotides 1-1274 of SEQ ID NO:4, a DNA fragment having nucleotides 291-1772 of SEQ ID NO:4, a DNA fragment having nucleotides 3387-3533 of SEQ ID NO:4, a DNA fragment having nucleotides 4704-5396 of SEQ ID NO:4, and a DNA fragment having nucleotides 4718-5437 of SEQ ID NO:4, wherein hybridization is conducted under the following conditions: a) hybridization: 0.75 M NaCl, 0.15 M Tris, 10 mM EDTA, 0.1% sodium pyrophosphate, 0.1% sodium lauryl sulfate, 0.03% BSA, 0.03% Ficoll 400, 0.03% PVP and 100 μg/ml boiled calf thymus DNA at 50° C. for about 12 hours and; b) wash: 3×30 minutes with 0.1X SET, 0.1% SDS, 0.1% sodium pyrophosphate and 0.1 M phosphate buffer at 45° C.

13. The method of claims 1-3 wherein the DNA polymerase is selected from the group consisting of Vent®, Deep Vent®, Pfu and 9° N™ DNA polymerase.

14. The method of claims 1-3 wherein the DNA polymerase has been mutated by substitution of the conserved amino acid residue corresponding to Vent® DNA polymerase A488, L492, A493 or Y499.

15. The method of claims 1-3 wherein the DNA polymerase has been mutated by substitution of the amino acid residue corresponding to Vent® DNA polymerase A488 by L, I. V, F, S or C.

16. The method of claims 1-3 wherein the DNA polymerase has been mutated by substituting the amino acid residue corresponding to Vent® DNA polymerase A488 by L.

17. The method of claims 1-3 wherein the DNA polymerase has been mutated by substitution of the amino acid residue corresponding to Vent® DNA polymerase Y499 to L.

18. The method of claims 1-3 wherein the DNA polymerase is selected from the group consisting of Vent® (A488L), Vent® (Y499L) and 9° N™ (A485L) DNA polymerases.

19. The method of claims 2 or 3 wherein the extent of acyclonucleotide incorporation is greater than that of the corresponding dideoxynucleotide.

20. The method of claims 2 or 3 wherein the extent of acyclonucleotide incorporation is at least, approximately, two-fold greater than incorporation of the corresponding dideoxynucleotide.

21. The method of claims 2 or 3 wherein the extent of acyclonucleotide incorporation is at least, approximately, five-fold greater than incorporation of the corresponding dideoxynucleotide.

22. The method of claims 2 or 3 wherein the extent of acyclonucleotide incorporation is at least, approximately, nine-fold greater than incorporation of the corresponding dideoxynucleotide.

23. The method of claims 2 or 3 wherein the extent of ROX-acyclo-CTP incorporation is greater than that of ROX-ddCTP.

24. The method of claims 2 or 3 wherein the extent of ROX-acyclo-CTP incorporation is at least, approximately, two-fold greater than that of ROX-ddCTP.

25. The method of claims 2 or 3 wherein the extent of ROX-acyclo-CTP incorporation is at least, approximately, five-fold greater than that of ROX-ddCTP.

26. The method of claims 2 or 3 wherein the extent of ROX-acyclo-CTP incorporation is at least, approximately, ten-fold greater than that of ROX-ddCTP.

27. The method of claims 1-3 wherein the DNA polymerase is additionally thermostable.

28. The method of claims 1-3 wherein the DNA polymerase has no detectable exonuclease activity.

29. The method of claims 1-3 wherein the DNA polymerase has less than about 5% of the exonuclease activity of the unmodified enzyme.

30. The method of claims 1-3 wherein the DNA polymerase has less than about 25% of the exonuclease activity of the unmodified enzyme.

31. The method of claims 1-3 further comprising the step of employing the resulting sequence-specific termination product or products in DNA sequence determination.
